SEQUENCE LISTING (7 sequences)

SEQ ID NO: 1 - DNA, 802 bp, Potato virus Y
  source 1..802: mol_type = genomic DNA
ggaaatgacacaattgatgcaggaggaagcactaagaaggatgcaaaacaagagctaggtagcattcaaccaaatctcaacaaggaaaaggaaaaggacgtgaatgttggaacatctggaactcatactgtgccacgaattaaagctatcacgtccaaaatgagaatgcccaagagtaaaggtgcaactgtactaaatttggaacacttactcgagtatgctccacagcaaattgacatctcaaatactcgagcaactcaatcacagtttgatacgtggtatgaagcggtacaacttgcatacggcataggagaaactgaaatgccaactgtgatgaatgggcttatggtttggtgcattgaaaatggaacctcgccaaacatcaacggagtttgggttatgatggatggagatgaacaagtcgaatacccactgaaaccaatcgttgagaatgcaaaaccaacacttaggcaaatcatggcacatttctcagatgttgcagaagcgtatatagaaatgcgcaacaaaaaggaaccatatatgccacgatatggtttagttcgtaatctgcgcgatggaagtttggctcgctatgcttttgacttttatgaggtcacatcacgaacaccagtgagggctagggaagcgcacattcaaatgaaggccgcagcattgaaatcagcccaacctcgacttttcgggttggacggtggcatcagtacacaagaggagaacacagagaggcacaccaccgaggatgtttctccaagtatgcatactctacttggagtcaagaacatgt

SEQ ID NO: 2 - DNA, 681 bp, synthetic construct
  source 1..681: mol_type = other DNA
atgaaccccatcgtaattaatcggcttcaacggaagctgggctacacttttaatcatcaggaactgttgcagcaggcattaactcatcgtagtgccagcagtaaacacaatgcccgcttggaatttttaggcgactctattctgagctacgttatcgccaatgcgctttatcaccgtttccctcgtgtggatgaaggcgatatgagccggatgcgcgccacgctggtccgtggcaatacgctggcggaactggcgcgcgaatttgagttaggcgagtgcttacgtttagggccaggtgaacttaaaagcggtggatttgccgccgagtcaattctcgccgacaccgtcgaagcattaattggtggcgtattcctcgacagtgatattcaaaccgtcgagaaattaatcctcaactggtatcaaactcgtttggacgaaattagcccaggcgataaacaaaaagatccgaaaacgcgcttgcaagaatatttgcagggtcgccatctgccgctgccgacttatctggtagtccaggtacgtggcgaagcgcacgatcaggaatttactatccactgccaggtcagcggcctgagtgaaccggtggttggcacaggttcaagccgtcgtaaggctgagcaggctgccgccgaacaggcgttgaaaaaactggagctggaatga

SEQ ID NO: 3 - protein, 226 aa, synthetic construct
MNPIVINRLQRKLGYTFNHQELLQQALTHRSASSKHNARLEFLGDSILSYVIANALYHRFPRVDEGDMSRMRATLVRGNTLAELAREFELGECLRLGPGELKSGGFAAESILADTVEALIGGVFLDSDIQTVEKLILNWYQTRLDEISPGDKQKDPKTRLQEYLQGRHLPLPTYLVVQVRGEAHDQEFTIHCQVSGLSEPVVGTGSSRRKAEQAAAEQALKKLELE

SEQ ID NO: 4 - DNA, 681 bp, synthetic construct
  source 1..681: mol_type = other DNA
atgaatcccatcgtaataaataggctgcagcgtaagctgggctacacttttcaacatcaggatctgttgcaacaggcattaacccatcggagtgccagcagcaagcataatgcccgcttggagtttttgggtgactccattctcagttatgtcatcgcgaatgcgctgtatcatcgttttcctcgcgtagatgaaggcgacatgagccgcatgcgtgcgacgctggtgcgcggcaatacgctggcggaaatcgcccgcgagttcgaactgggtgagtgtctgcgtcttgggccgggtgaactgaaaagtggcggtttcgccgccgagtcgattcttgctgataccgtggaagcgttgatcggtggcgtcttcctcgacagcgacattcagaacgttgagcgtttgattctctcgtggtatcagacccgtctcgacgaaatcagtccaggcgacaagcaaaaagatccgaaaacgcgtctgcaggagtacctgcagggtcgccatctgccgctgccgtcgtatctggtggtgcaggtgcgtggtgaagcgcacgatcaagaatttaccattcactgtcaggtgagtggcctgcctgagcctgtcgtagggacgggctcaagccgccgtaaagcggaacaggctgcggctgagcaggcactgaaaaagctggagctggaatga

SEQ ID NO: 5 - protein, 226 aa, synthetic construct
MNPIVINRLQRKLGYTFQHQDLLQQALTHRSASSKHNARLEFLGDSILSYVIANALYHRFPRVDEGDMSRMRATLVRGNTLAEIAREFELGECLRLGPGELKSGGFAAESILADTVEALIGGVFLDSDIQNVERLILSWYQTRLDEISPGDKQKDPKTRLQEYLQGRHLPLPSYLVVQVRGEAHDQEFTIHCQVSGLPEPVVGTGSSRRKAEQAAAEQALKKLELE

SEQ ID NO: 6 - DNA, 681 bp, synthetic construct
  source 1..681: mol_type = other DNA
atgaaccccatcgtaattaatcggcttcaacggaagctgggctacacttttcatcatcaggagttgttgcaacaggcattaacccaccgcagtgccagcagcaagcacaacgcccgcctggagtttttaggcgactctattttaagtttcgtgattgcgaatgcgctttatcatcgtttcccgcgcgtggatgaaggtgatatgagccgcatgcgtgccacgctggttcggggtaacacccttgcggaaatcgcgcgcgaatttgaactgggcgaatgtctgcgtcttgggccgggtgaactgaaaagcggcggcttcgccgccgaatctattcttgccgatacggtcgaagcattaattggtggtgtgttcctggacagcgatatccagaccgtcgaaaagctgatcctgaactggtatcagacccgtctggacgaaatcagcccgggcgataaacaaaaagatcccaaaacgcgtctgcaggaatatttgcagggccgtcatctgccgctgccatcttatctggtggtgcaggttcgtggcgaagcgcacgatcaggaatttaccatccattgccaggtcagtggcctgagtgaaccggtggtgggcacaggttcaagccgtcgtaaggctgaacaggctgccgccgaacaggcgttaaaaatgctggagctggaatga

SEQ ID NO: 7 - protein, 226 aa, synthetic construct
MNPIVINRLQRKLGYTFHHQELLQQALTHRSASSKHNARLEFLGDSILSFVIANALYHRFPRVDEGDMSRMRATLVRGNTLAEIAREFELGECLRLGPGELKSGGFAAESILADTVEALIGGVFLDSDIQTVEKLILNWYQTRLDEISPGDKQKDPKTRLQEYLQGRHLPLPSYLVVQVRGEAHDQEFTIHCQVSGLSEPVVGTGSSRRKAEQAAAEQALKMLELE

(19) United States
(12) Patent Application Publication
(10) Pub. No.: US 2023/0225331 A1
(43) Pub. Date: Jul. 20, 2023

(54) Plant Endophytic Bacteria And Methods To Control Plant Pathogens And Pests

(71) Applicant: Pebble Labs, Inc., Los Alamos, NM (US)

(72) Inventors: Shujian Zhang, Chapel Hill, NC (US); Rick DeRose, Santa Fe, NM (US); Rosa Otero, Los Alamos, NM (US)

(21) Appl. No.: 17/822,349

(22) Filed: Aug. 25, 2022

Related U.S. Application Data
(63) Continuation-in-part of application No. PCT/US2021/019847, filed on Feb. 26, 2021.
(60) Provisional application No. 62/981,705, filed on Feb. 26, 2020.

(51) Int. Cl. A01N 63/27; A01H 3/00; C12N 1/20

(52) U.S. Cl. CPC A01N 63/27; A01H 3/00; C12N 1/20

(57) ABSTRACT

The inventive technology generally relates to novel plant endophytic bacterial strains that may colonize discrete plant tissues, and in particular the roots of a plant and may further be engineered to express and deliver interfering RNA molecules throughout the plant.

This application is a continuation-in-part which claims priority to, and the benefit of International PCT Application PCT/US2021/019847, filed Feb. 26, 2021, claims the benefit of and priority to U.S. Provisional Application No. 62/981,705, filed Feb. 26, 2020, which is incorporated herein by reference in its entirety.

### SEQUENCE LISTING

The instant application contains contents of the electronic sequence listing submitted electronically as a Standard ST.26 compliant XML file, entitled “58198-US.xml,” created on Feb. 28, 2023, as 10,587 bytes in size, the content of which is herein incorporated by reference in its entirety.

### TECHNICAL FIELD

The inventive technology generally relates to novel plant endophytic bacterial strains that may colonize discrete plant tissues, and in particular the roots of a plant and may further be engineered to express and deliver interfering RNA molecules throughout the plant.

### BACKGROUND

A major limiting factor in the development of genetically modified plants has been the difficult and extended process required for making safe, effective, and importantly, stable transgenic expression systems. To develop even one transgenic crop plant may take up to ten years and requires significant financial investment. For example, the development of a transgenic strain may be limited by the ability to successfully reproduce seeds or allow the natural outgrowth and spread of the transgenic plant species in any given environment. In addition, significant social pressures have arisen in opposition to the use of transgenic plants. Movements in Western and European countries have sought to prevent the use of transgenic plants in food stuffs. Additionally, new markets for organic and non-transgenic products have concurrently arisen in the last decade which allows non-transgenic plants to be sold at a market premium. Finally, many transgenic plants are controlled by large agricultural conglomerates making supply of seeds for transgenic plants limited, and thereby inhibiting their widespread use in many third-world countries, as one example. In an effort to address some of the limitations, plant-based microorganisms, such as endophytes and the like, have been proposed as delivery vectors for beneficial genes, as well as genetic inhibition of undesirable genes, such as genes expressed by plant pathogens.

As will be discussed in more detail below, the current inventive technology includes the novel use endophytic bacteria to provide a vehicle for stable and continuous non-integrative transformation of a plant host cell through the continual delivery of select molecules, such as RNA molecules configured to induce an endogenous RNA interference response, as well as mRNAs produced in prokaryotic organisms that are configured to be translated in a plant host cell.

### SUMMARY OF THE INVENTION

One aspect of the current invention includes isolation and identification of novel botanically compatible plant botanically compatible endophytic bacteria. As used herein, the term “botanically compatible” means a plant endophyte that is culturable, transformable, and persists in at least one discrete tissue of a target plant, such as the roots. In one preferred embodiment, the current invention includes isolation and identification of novel botanically compatible plant botanically compatible endophytic bacteria persists in the roots of a target plant host.

Another aspect of the current invention includes isolation and identification of novel botanically compatible plant botanically compatible endophytic bacteria. In one preferred aspect, the current invention includes isolation and identification of novel botanically compatible plant botanically compatible endophytic bacteria that persists in the roots of a target plant host, and not any other discrete plant tissues, such as the leaves, stems, flowers, or fruit. One aim of the current invention includes isolation and identification of plant endophyte which could be culturable transformable. In one aspect, the invention may include plant endophytes Pseudomonas sp. Csr-7 as deposited with the ATCC, and Csr-8 as deposited with the ATCC.

Another aspect of the current invention related to plant endophytic bacteria or bacterial combinations that may be used in methods to control plant pathogens, nematodes, and pests. In one aspect, the inventive technology includes endophytic bacteria that may selectively colonize one or more plant tissues. In one preferred aspect, the botanically compatible endophytic bacteria may selectively colonize the plant roots only.

In another aspect, the present invention relates to novel systems, methods and compositions for the biocontrol of plant pathogens and pests. In one preferred aspect, the present invention may include novel genetically modified plant endophytic bacteria, or novel bacterial combinations, which can be engineered to express one or more heterologous inhibitory RNA molecules that may be delivered to the target host plant.

In another aspect, the inventive technology may include compositions and methods of use for the endophytic bacterial strains Pseudomonas sp. Csr-7 and Pseudomonas sp. Csr-8. In one aspect, endophytic bacterial strains Csr-7 and Csr-8 may be genetically engineered to express a heterologous nucleotide sequence, operably linked to a promoter sequence, encoding an inhibitory RNA molecule. In this preferred aspect, endophytic bacterial strains Csr-7 and Csr-8 may be genetically engineered to express a heterologous nucleotide sequence, operably linked to a promoter sequence, encoding one or more double stranded RNA (dsRNA) molecules directed to initiate a DICER-mediated RNA interference (RNAi) response causing the destruction of specifically targeted pathogen, nematode, or pest mRNA molecules that may be present within a plant host. In further aspects, the endophytic bacteria such as Pseudomonas sp. Csr-7 and Csr-8 may be localized in the roots of a plant, such that the dsRNAs are delivered to the target plant host and distributed throughout the plant's intracellular architecture such as the plant's the leaves, fruit, and stems.

In another aspect, the present invention may include novel genetically modified plant endophytic bacteria, or novel bacterial combinations, which can be used to deliver one or more heterologous dsRNA molecules thereby initiating an RNAi response in a target host plant. In one preferred aspect, the plant endophytes, and preferably Pseudomonas sp. Csr-7 and Csr-8, may be configured to express a heterologous dsRNA configured to inhibit expression of one or more essential genes of a plant or plant pathogen. The plant endophytes, and preferably Pseudomonas sp. Csr-7 and Csr-8, may be further configured to express a heterologous engineered RNase III enzyme configured to processes double-strand RNA (dsRNA) almost exclusively into small RNAs (sRNAs) comprising of 22 and 23 nt. These sRNAs may be transported from the bacterial strains and initiate a DICER-independent RNAi response in a target host plant. In one preferred aspect, the engineered RNase III may include an engineered RNase III having the following mutations E38A/R107A/R108A (RNase III N3XT™ or mc) which was demonstrated to processes double-strand RNA (dsRNA) almost exclusively into sRNAs (sRNAs) comprising of 22 and 23 nt (such systems, methods and compositions, being included in Sayre et al., PCT/US2019/025261, the specification, sequences 1-121, examples, and figures related to the generation of siRNAs in a DICER independent manner are herein incorporated in their entirety by reference).

In another aspect, Pseudomonas sp. Csr-7 and Csr-8 expressing an dsRNA targeting one or more essential genes in a plant pathogen, nematode or pest, and an engineered RNase III mutant having the following mutations E38A/R107A/R108A can be useful for the treatment of plants which are susceptible to pathogens such as viral pathogens, fungal pathogens, and bacterial pathogens as well as nematodes and insects. The plants could be those commercial crops like potato, corn, soybean, potato and citrus and even treatment on the seeds of the above crops.

The present invention related to plant endophytic bacteria or bacterial combinations that may be used in methods to inhibit expression of one or more endogenous plant genes. In one aspect the, inventive technology includes endophytic bacteria that may selectively colonize one or more plant tissues. In one preferred aspect, the botanically compatible endophytic bacteria may selectively colonize the plant roots only and that may further be configured to inhibit expression of one or more endogenous plant genes throughout the entire plant. In one preferred aspect, the present invention may include novel genetically modified plant endophytic bacteria, or novel bacterial combinations, which can be used to deliver one or more heterologous inhibitory RNA molecules to the plant that are configured to inhibit expression of one or more endogenous plant genes.

In another aspect, the inventive technology may include compositions and methods of use for the endophytic bacterial strains Pseudomonas sp. Csr-7 and Pseudomonas sp. Csr-8. In one aspect, endophytic bacterial strains Csr-7 and Csr-8 may be genetically engineered to express a heterologous nucleotide sequence, operably linked to a promoter sequence, encoding an inhibitory RNA molecule. In this preferred aspect, endophytic bacterial strains Csr-7 and Csr-8 may be genetically engineered to express a heterologous nucleotide sequence, operably linked to a promoter sequence, encoding one or more double stranded RNA (dsRNA) molecules directed to initiate a DICER-mediated RNA interference (RNAi) response causing the destruction of specifically targeted endogenous mRNA molecules within the plant host. In further aspects, the endophytic bacteria such as Pseudomonas sp. Csr-7 and Csr-8 may be localized in the roots of a plant, such that the siRNAs are delivered to the target host plant and distributed throughout the plant's intracellular architecture such as the plant's leaves, fruit, and stems.

In another aspect, the present invention may include novel genetically modified plant endophytic bacteria, or novel bacterial combinations, which can be used to deliver one or more heterologous dsRNA molecules thereby initiating an RNAi response directed to an endogenous gene in a target host plant. In one preferred aspect, the plant endophytes, and preferably Pseudomonas sp. Csr-7 and Csr-8, may be configured to express a heterologous dsRNA configured to inhibit expression of one or more endogenous host genes. The plant endophytes, and preferably Pseudomonas sp. Csr-7 and Csr-8, may be further configured to express a heterologous engineered RNase III enzyme configured to processes double-strand RNA (dsRNA) almost exclusively into small RNAs (sRNAs) comprising of 22 and 23 nt. These sRNAs may be transported from the bacterial strains and initiate a DICER-independent RNAi response directed to an endogenous gene in a target host plant, or a gene of a plant pathogen.

In another aspect, the present invention may include plant endophytic bacteria, or bacterial combinations, that may be engineered to express one or more RNA transcripts that may be delivered and translated in a target plant host. In one preferred aspect, plant endophytic bacteria may be configured to produce eukaryotic-like mRNA that may be introduced to, and translated in a plant host, for example as generally described by Sayre et al in PCT/US2019/040747 (the specification, figures, and sequences SEQ ID NOs. 1-37 being specifically incorporated herein by reference).

In one aspect, the inventive technology includes endophytic bacteria configured to produce eukaryotic-like mRNA that may be introduced to and translated in a plant host that may selectively colonize one or more plant tissues. In one preferred aspect, the botanically compatible endophytic bacteria may selectively colonize the plant roots only and that may further be configured to produce eukaryotic-like mRNA that may be introduced to and translated throughout the entire plant. In one preferred aspect, the present invention may include novel genetically modified plant endophytic bacteria, or novel bacterial combinations, which can be used to express and deliver one or more eukaryotic-like mRNA to the plant that are configured to be translated by the plant such that the eukaryotic-like mRNA may introduce a novel heterologous protein into the plant, or in other aspect, increase production of an endogenous protein.

In another aspect, the inventive technology may include compositions and methods of use for the endophytic bacterial strains Pseudomonas sp. Csr-7 and Pseudomonas sp. Csr-8. In one aspect, endophytic bacterial strains Csr-7 and Csr-8 may be genetically engineered to express a heterologous nucleotide sequence, operably linked to a promoter sequence, encoding one or more eukaryotic-like mRNA. In this preferred aspect, endophytic bacterial strains Csr-7 and Csr-8 may be genetically engineered to express a heterologous nucleotide sequence, operably linked to a promoter sequence, encoding one or more eukaryotic-like mRNA that may be transported and translated throughout the plant host. In further aspects, the endophytic bacteria such as Pseudomonas sp. Csr-7 and Csr-8 may be localized in the roots of a plant, such that the one or more eukaryotic-like mRNAs may be distributed throughout the plant's intracellular architecture for example, to the plant's leaves, fruit, and stems.

In another aspect, the present invention may include novel genetically modified plant endophytic bacteria, or novel bacterial combinations, which can be used to deliver one or more heterologous dsRNA molecules thereby initiating an RNAi response directed to a pathogen gene, or endogenous gene in a target host plant, and a eukaryotic-like mRNA that may be introduced to and translated in the plant host. In one preferred aspect, the plant endophytes, and preferably Pseudomonas sp. Csr-7 and Csr-8, may be configured to express a heterologous dsRNA configured to inhibit expression of a pathogen gene, or endogenous gene in a target host plant, and a eukaryotic-like mRNA that may be introduced to and translated in the plant host. The plant endophytes, and preferably Pseudomonas sp. Csr-7 and Csr-8, may be further configured to express a heterologous engineered RNase III enzyme configured to processes double-strand RNA (dsRNA) almost exclusively into small RNAs (sRNAs) comprising of 22 and 23 nt. These sRNAs may be transported from the bacterial strains and initiate a DICER-independent RNAi response directed to an endogenous gene in a target host plant or a gene of a plant pathogen.

Another aspect of the invention may include the isolated bacteria Pseudomonas sp. Csr-7, and Csr-8.

In one aspect, the present invention relates to novel systems, methods and compositions for the biocontrol of a plant viral pathogen. In one preferred aspect, the present invention may include novel genetically modified plant endophytic bacteria, or novel bacterial combinations, which can be used in the biocontrol of a plant viral pathogen through the delivery of heterologous inhibitory RNA molecules.

Another aspect of the current invention includes the expression and delivery of inhibitory RNAs homologous to the viral genome to efficiently down-regulate or eliminate viral replication and translation of viral proteins of a plant viral pathogen. In one preferred aspect, the inhibitory RNAs may be dsRNA configured to initiate a DICER-mediated RNA interference response in the target plant. In another preferred aspect, the inhibitory RNAs may be dsRNA, further processed in a bacterial host cell by an engineered RNaseIII configured to generate siRNAs that may be delivered to the target plant and initiate a DICER-independent RNA interference response in the target plant. In another aspect of the current invention, an engineered RNaseIII configured to generate siRNAs may include novel E. coli RNase III having the following mutations E38A/R107A/R108A (RNase III N3XT™), which in this preferred aspect may be expressed in planta endophytic bacteria and deliver 22-23 nt siRNAs to a target plant host. In this preferred aspect, the 22-23 nt interfering RNAs may be generated from a co-expressed dsRNA molecules.

Another aspect of the current invention includes isolation and identification of novel botanically compatible plant endophytic bacteria which may be culturable, transformable, and persists only in roots of a plant and that further express one or more heterologous inhibitory RNA molecules, such as a double stranded RNA (dsRNA) molecule that may be configured to initiate an RNA interference response in a target plant host. In this preferred aspect, the genetically engineered plant endophytic bacteria may persist in the plant's roots and express and transport dsRNA molecules that may be configured to initiate an RNA interference response in a target plant. The dsRNA molecules may preferably be hairpin RNA (hpRNA) molecules that may be produced by the endophytic bacteria in the roots and distributed to the rest of the plant through the plant's intracellular architecture.

Another aspect of the current invention includes methods of against, and/or treating a pathogen in a plant, or seed. In one preferred aspect, this method may include the isolation and identification of novel botanically compatible plant endophytic bacteria which may be culturable, transformable, and persists only in roots of a plant and that further express one or more heterologous inhibitory RNA molecules, such as a double stranded RNA (dsRNA) molecule that may be configured to initiate an RNA interference response in a target plant host. In this preferred aspect, the genetically engineered plant endophytic bacteria may persist in the plant's roots and express and transport dsRNA molecules that may be configured to initiate an RNA interference response in a target plant. The dsRNA molecules may preferably be hairpin RNA (hpRNA) molecules that may be produced by the endophytic bacteria in the roots and distributed to the rest of the plant through the plant's intracellular architecture.

Another aspect of the current invention includes methods of inoculating against, and/or treating a pathogen in a plant, or seed. In one preferred aspect, this method may include the isolation and identification of novel botanically compatible plant endophytic bacteria which may be culturable, transformable, and persists in a discrete plant tissue and that further express one or more heterologous inhibitory RNA molecules, such as a double stranded RNA (dsRNA) molecule that may be configured to initiate an RNA interference response in a target plant host. In this preferred aspect, the genetically engineered plant endophytic bacteria may persist in the plant express and transport dsRNA molecules that may be configured to initiate an RNA interference response in a target plant. The dsRNA molecules may preferably be hairpin RNA (hpRNA) molecules that may be produced the endophytic bacteria in, or on, the plant and distributed to the rest of the plant through the plant's intracellular architecture. Another aspect of the current invention includes the production and isolation of dsRNAs, or siRNAs produced by an engineered RNase enzyme as herein described and administered to a plant that is susceptible to or infected with a plant viral pathogen.

In another aspect, a plant may be inoculated at the root level and remain free of genetically modified organisms throughout the rest of the plant, such as the stems, leaves and fruit. In another aspect, a plant may be inoculated at the root level and remain free of genetically modified organisms throughout the rest of the plant, such as the stems, leaves and fruit, but the entire plant may be treated or inoculated against a viral pathogen, such as a plant viral pathogen, through the distribution of dsRNA molecules throughout the plant. In this manner, the plant may be treated or inoculated against a viral pathogen, such as a plant viral pathogen, without requiring the stable genetic transformation and propagation of the plant. This non-GMO strategy may allow the present solution to the treatment of a plant viral pathogen to employ a non-GMO aspect, as well as the ability to rapidly modify one or more dsRNA constructs to adapt to any changes in the target essential genes sequence.

In another aspect, a plant may be inoculated with a genetically modified endophytic bacteria that expresses a heterologous RNA interference molecule, such as a hpRNA, directed to one or more a plant viral pathogen genes, and a heterologous engineered RNaseIII enzyme configured to process the hpRNA molecules into 22-23 nt small interfering RNAs in the bacteria. In this aspect, the genetically modified endophytic bacteria may be configured to, or naturally be maintained, at the root level such that the remaining tissues of the plant, such as the plant, such as the stems, leaves and fruit, remain free of genetically modified organisms throughout the lifecycle of the plant. In another aspect, a plant may be inoculated at the root level and there express one or more heterologous RNA interference molecule, such as a hpRNA, directed to one or more a plant viral pathogen genes, and a heterologous engineered RNaseIII enzyme configured to process the hpRNA molecules into 22-23 nt siRNAs in the bacteria that may be delivered to the target plant's roots, and distributed through the rest of the plant. In this preferred aspect, the non-root plant tissues may remain free of genetically modified organisms, such as the stems, leaves and fruit, but the entire plant may be treated or inoculated against a pathogen, such as a viral pathogen or pest, through the production and distribution of siRNA molecules generated by the engineered RNaseIII and delivered throughout the plant from the root tissues. In this manner, the plant may be treated or inoculated against a viral pathogen, such as a plant viral pathogen, and/or pest, without requiring the stable genetic transformation and propagation of the plant.

Another aspect of the current invention includes methods of against, and/or treating a pathogen or pest in a plant, or seed. In one preferred aspect, this method may include the identification of novel botanically compatible plant endophytic bacteria that may express one or more heterologous inhibitory RNA molecules, such as a double stranded RNA (dsRNA) molecule that may be configured to initiate an RNA interference response in a target plant host, as well as a heterologous engineered RNaseIII enzyme configured to process the dsRNA molecules into 22-23 nt interfering RNAs in the bacteria that may further be delivered to the tissues of a plant and initiate an RNAi response, and preferably an RNAi response targeting one or more essential genes in a pathogen or pest.

Another aspect of the current invention includes methods of inoculating against, and/or treating a pathogen or pest in a plant, or seed. In one preferred aspect, this method may include the inoculating a plant susceptible to, or infected with a pathogen or pest with a genetically modified endophytic bacteria that expresses a heterologous RNA interference molecule, such as a hpRNA, directed to one or more pathogen or pest genes, and a heterologous engineered RNaseIII enzyme configured to process the hpRNA molecules into 22-23 nt small interfering RNAs in the bacteria that may be delivered to the plant or seed thereby initiating a DICER-independent RNAi response targeting one or more essential genes in a pathogen or pest in the plant or seed. The siRNA molecules may preferably be distributed to the rest of the plant through the plant's intracellular architecture.

In this preferred aspect, the genetically engineered plant endophytic bacteria may persist in the plant's roots and be vertically and/or horizontally transferred to its progeny or other plants where the genetically engineered plant endophytic bacteria may express and transport dsRNA molecules that may be configured to initiate an RNA interference response in a target plant host. The dsRNA molecules may preferably be hairpin RNA (hpRNA) molecules and may further be processed an engineered RNaseIII protein as generally described herein to generate siRNA that may initiate a DICER-independent RNA interference response in a target plant.

In this preferred aspect, the genetically engineered plant endophytic bacteria may persist in the plant's roots and be vertically and/or horizontally transferred to its progeny or other plants where the genetically engineered plant endophytic bacteria may express and transport dsRNA molecules that may be configured to initiate an RNA interference response in a target plant. The dsRNA molecules may preferably be hairpin RNA (hpRNA) molecules and may further be processed an engineered RNaseIII protein as generally described herein to generate siRNA that may initiate a DICER-independent RNA interference response in a target plant.

Additional aspects of the invention may become evident in light of the figures and disclosure provided below.

### BRIEF DESCRIPTION OF THE FIGURES

FIG. 1. The colonies of Csr-7/GFP were re-isolated from potato roots but from potato tubers at 82 days post inoculation (dpi). The concentration of Csr-7/GFP reached to 2.09×10³ cfu/g tissue.

FIG. 2. The colonies of Csr-7/GFP were re-isolated from the roots of potato “Moneymaker” 117 days post inoculation (dpi). The concentration of Csr-7/GFP reached to 7.7×10² cfu/g tissue. The bacteria were not found in stems, upper branches, flower or fruits.

FIG. 3. The colonies of Csr-8/GFP were re-isolated from the roots of potato “Russet Burbank” at 105 days post inoculation (dpi). The bacteria were not found in stems, upper branches, flower or fruits.

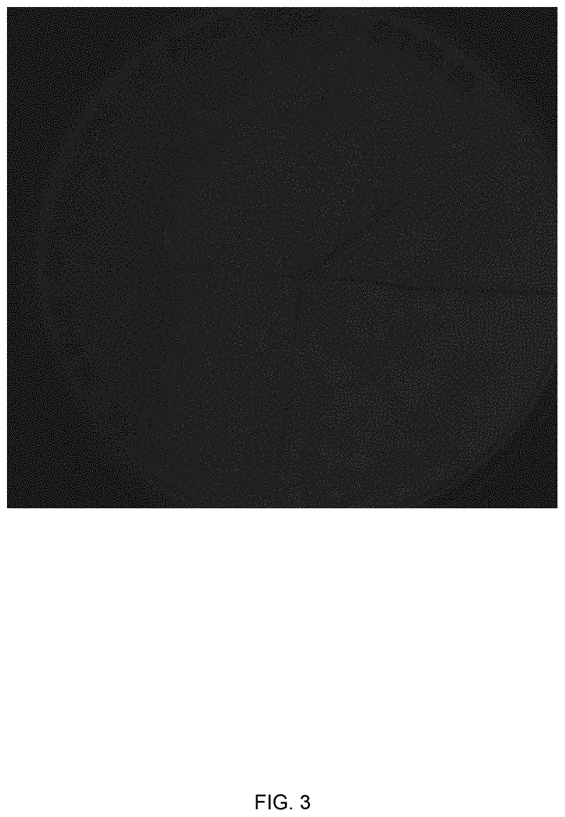

FIG. 4. Structural model of two E. coli E38A/R107A/R108A mutant RNase III dimers (green and blue cartoons) separated by 22 nt along a dsRNA target. Note that at this separation, both dimers are not showing steric clashes with each other, as indicated by the absence of any overlaps between their van der Waals (i.e., molecular) surfaces. Yellow and red RNA strands represent the 22-nt long dsRNA cleavage product, while white RNA strands denote the rest of the dsRNA target.

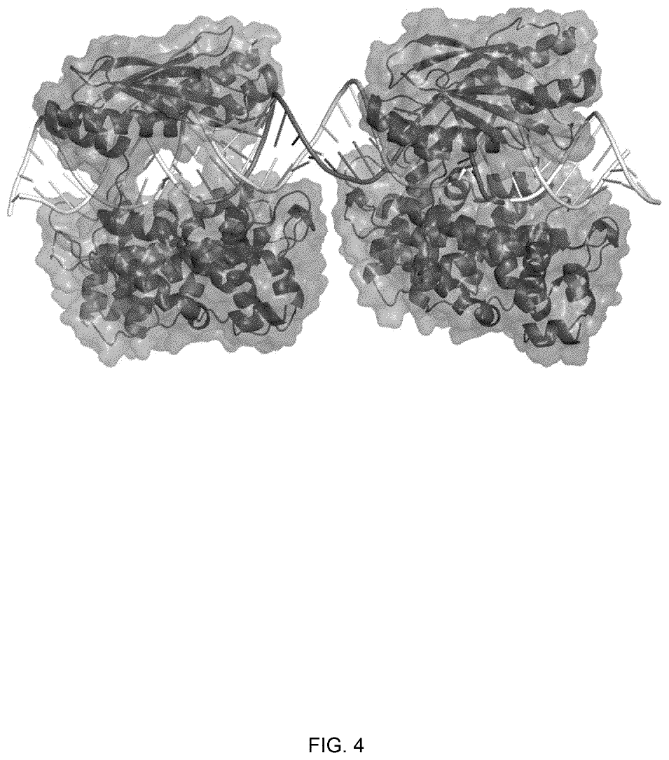

FIG. 5. API 20NE test strip after 48-hrs for Csr-7 (A) and Csr-8 (B). Results correspond to Table 1

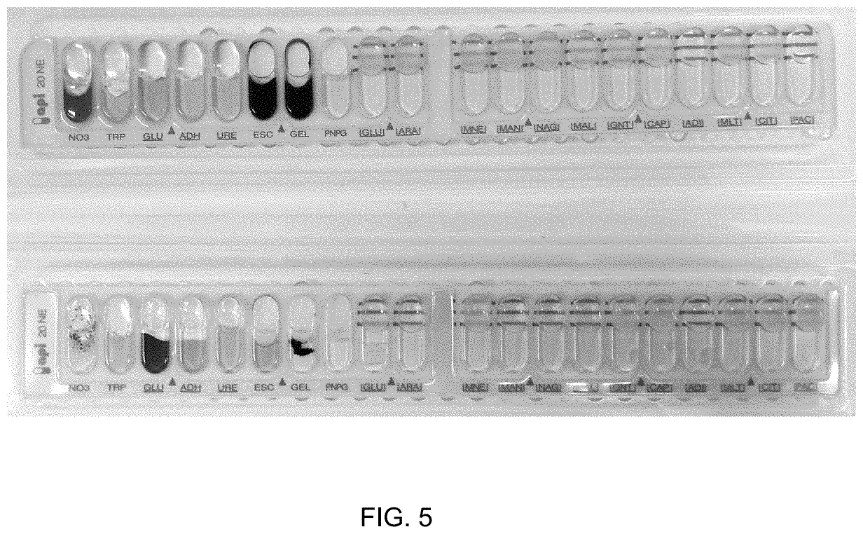

FIG. 6. Oxidase test results: positive reactions for both Csr-7 and Csr-8 are indicated by purple color.

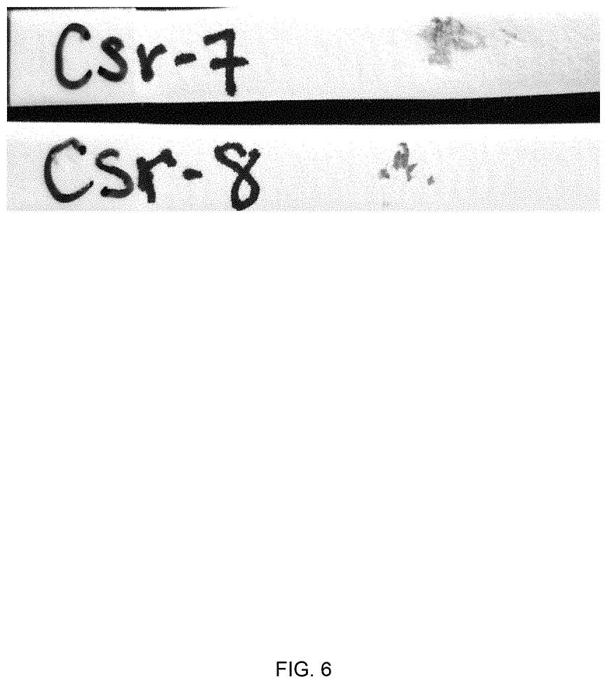

### DETAILED DESCRIPTION OF THE INVENTION

The present invention includes isolation and identification of novel botanically compatible endophytic bacteria. Another embodiment of the current invention related to plant endophytic bacteria or bacterial combinations that may be used in methods to control plant pathogens, nematodes, and pests. In one embodiment the inventive technology includes endophytic bacteria that may selectively colonize one or more plant tissues. In another preferred embodiment, the botanically compatible endophytic bacteria may selectively colonize the plant roots only. In another embodiment, the present invention relates to novel systems, methods and compositions for the biocontrol of plant pathogens and pests. In one preferred embodiment, the present invention may include novel genetically modified plant endophytic bacteria, or novel bacterial combinations, that can be used to deliver one or more heterologous inhibitory RNA molecules to the plant.

In another embodiment, the inventive technology may include compositions and methods of use for the endophytic bacterial strains Pseudomonas sp. Csr-7 and Pseudomonas sp. Csr-8. In one embodiment, endophytic bacterial strains Csr-7 and Csr-8 may be genetically engineered to express a heterologous nucleotide sequence, operably linked to a promoter sequence, encoding an inhibitory RNA molecule. In this preferred embodiment, endophytic bacterial strains Csr-7 and Csr-8 may be genetically engineered to express a heterologous nucleotide sequence, operably linked to a promoter sequence, encoding one or more double stranded RNA (dsRNA) molecules directed to initiate a DICER-mediated RNA interference (RNAi) response causing the destruction of specifically targeted pathogen, nematode, or pest mRNA molecules that may be present within a target plant host or in a pest or herbivore that consumes the target plant host. In further embodiments, the endophytic bacteria such as Pseudomonas sp. Csr-7 and Csr-8 may be localized in the roots of a plant, such that the dsRNAs are delivered to the target plant and distributed throughout the plant's intracellular architecture such as the plant's leaves, fruit, and stems.

In another embodiment, the present invention may include novel genetically modified plant endophytic bacteria, or novel bacterial combinations, which can be used to deliver one or more heterologous dsRNA molecules thereby initiating an RNAi response in a target host plant. In one preferred embodiment, the plant endophytes, and preferably Pseudomonas sp. Csr-7 and Csr-8, may be configured to express a heterologous dsRNA configured to inhibit expression of one or more essential genes of a plant pathogen. The plant endophytes, and preferably Pseudomonas sp. Csr-7 and Csr-8, may be further configured to express a heterologous engineered RNase III enzyme configured to processes double-strand RNA (dsRNA) almost exclusively into small RNAs (sRNAs) comprising of 22 and 23 nt. These sRNAs may be transported from the bacterial strains and initiate a DICER-independent RNAi response in a target host plant, nematode, pest, or herbivore that may consume the target plant host. In one preferred embodiment, the engineered RNase III may include an engineered RNase III having the following mutations E38A/R107A/R108A (RNase III N3XT™ or mc) which was demonstrated to processes double-strand RNA (dsRNA) almost exclusively into sRNAs (sRNAs) comprising of 22 and 23 nt (such systems, methods and compositions, being included in Sayre et al., PCT/US2019/025261, the specification, sequences 1-121, examples, and figures related to the generation of siRNAs in a DICER independent manner are herein incorporated in their entirety by reference).

In another embodiment, Pseudomonas sp. Csr-7 and Csr-8 expressing an dsRNA targeting one or more essential genes in a plant pathogen, nematode, pest, or herbivore, and an engineered RNase III mutant having the following mutations E38A/R107A/R108A can be usefully for the treatment of plants which are susceptible to pathogens such as viral pathogens, fungal pathogens, and bacterial pathogens as well as nematodes and insects. The plants could be those commercial crops like potato, corn, soybean, potato and citrus and even treatment on the seeds of the above crops.

The present invention related to plant endophytic bacteria or bacterial combinations that may be used in methods to inhibit expression of one or more endogenous plant genes. In one embodiment the inventive technology includes endophytic bacteria that may selectively colonize one or more plant tissues. In one preferred embodiment, the botanically compatible endophytic bacteria may selectively colonize the plant roots only and that may further be configured to inhibit expression of one or more endogenous plant genes throughout the entire plant. In one preferred embodiment, the present invention may include novel genetically modified plant endophytic bacteria, or novel bacterial combinations, which can be used to deliver one or more heterologous inhibitory RNA molecules to the plant that are configured to inhibit expression of one or more endogenous plant genes.

In another embodiment, the inventive technology may include compositions and methods of use for the endophytic bacterial strains Pseudomonas sp. Csr-7 and Pseudomonas sp. Csr-8. In one embodiment, endophytic bacterial strains Csr-7 and Csr-8 may be genetically engineered to express a heterologous nucleotide sequence, operably linked to a promoter sequence, encoding an inhibitory RNA molecule. In this preferred embodiment, endophytic bacterial strains Csr-7 and Csr-8 may be genetically engineered to express a heterologous nucleotide sequence, operably linked to a promoter sequence, encoding one or more double stranded RNA (dsRNA) molecules directed to initiate a DICER-mediated RNA interference (RNAi) response causing the destruction of specifically targeted endogenous mRNA molecules within the plant host. In further embodiments, the endophytic bacteria such as Pseudomonas sp. Csr-7 and Csr-8 may be localized in the roots of a plant, such that the siRNAs are delivered to the target host plant and distributed throughout the plant's intracellular architecture such as the plant's leaves, fruit, and stems.

In another embodiment, the present invention may include novel genetically modified plant endophytic bacteria, or novel bacterial combinations, which can be used to deliver one or more heterologous dsRNA molecules thereby initiating an RNAi response directed to an endogenous gene in a target host plant. In one preferred embodiment, the plant endophytes, and preferably Pseudomonas sp. Csr-7 and Csr-8, may be configured to express a heterologous dsRNA configured to inhibit expression of one or more endogenous host genes. The plant endophytes, and preferably Pseudomonas sp. Csr-7 and Csr-8, may be further configured to express a heterologous engineered RNase III enzyme configured to processes double-strand RNA (dsRNA) almost exclusively into small RNAs (sRNAs) comprising of 22 and 23 nt. These sRNAs may be transported from the bacterial strains and initiate a DICER-independent RNAi response directed to an endogenous gene in a target host plant.

The present invention related to plant endophytic bacteria or bacterial combinations that may be used to express one or more RNA transcripts that may be translated in a target plant host. In one preferred embodiment, plant endophytic bacteria may be configured to produce eukaryotic-like mRNA that may be introduced to and translated in a plant host (as generally described by Sayre et al in PCT/US2019/040747, the specification, figures, and sequences SEQ ID NOs. 1-37 being specifically incorporated herein by reference).

In one embodiment the inventive technology includes endophytic bacteria configured to produce eukaryotic-like mRNA that may be introduced to and translated in a plant host that may selectively colonize one or more plant tissues. In one preferred embodiment, the botanically compatible endophytic bacteria may selectively colonize the plant roots only and that may further be configured to produce eukaryotic-like mRNA that may be introduced to and translated throughout the entire plant. In one preferred embodiment, the present invention may include novel genetically modified plant endophytic bacteria, or novel bacterial combinations, which can be used to express and deliver one or more eukaryotic-like mRNA to the plant that are configured to be translated by the plant such that the eukaryotic-like mRNA may introduce a novel heterologous protein into the plant, or in other embodiment, increase production of an endogenous protein.

In another embodiment, the inventive technology may include compositions and methods of use for the endophytic bacterial strains Pseudomonas sp. Csr-7 and Pseudomonas sp. Csr-8. In one embodiment, endophytic bacterial strains Csr-7 and Csr-8 may be genetically engineered to express a heterologous nucleotide sequence, operably linked to a promoter sequence, encoding one or more eukaryotic-like mRNA. In this preferred embodiment, endophytic bacterial strains Csr-7 and Csr-8 may be genetically engineered to express a heterologous nucleotide sequence, operably linked to a promoter sequence, encoding one or more eukaryotic-like mRNA that may be transported and translated throughout the plant host. In further embodiments, the endophytic bacteria such as Pseudomonas sp. Csr-7 and Csr-8 may be localized in the roots of a plant, such that the one or more eukaryotic-like mRNAs may be distributed throughout the plant's intracellular architecture for example, to the plant's leaves, fruit, and stems.

In another embodiment, the present invention may include novel genetically modified plant endophytic bacteria, or novel bacterial combinations, which can be used to deliver one or more heterologous dsRNA molecules thereby initiating an RNAi response directed to a pathogen gene, or endogenous gene in a target host plant, and a eukaryotic-like mRNA that may be introduced to and translated in the plant host. In one preferred embodiment, the plant endophytes, and preferably Pseudomonas sp. Csr-7 and Csr-8, may be configured to express a heterologous dsRNA configured to inhibit expression of a pathogen gene, or endogenous gene in a target host plant, and a eukaryotic-like mRNA that may be introduced to and translated in the plant host. The plant endophytes, and preferably Pseudomonas sp. Csr-7 and Csr-8, may be further configured to express a heterologous engineered RNase III enzyme configured to processes double-strand RNA (dsRNA) almost exclusively into small RNAs (sRNAs) comprising of 22 and 23 nt. These sRNAs may be transported from the bacterial strains and initiate a DICER-independent RNAi response directed to an endogenous gene in a target host plant.

Another embodiment of the invention may include isolated bacteria Pseudomonas sp. Csr-7, and Csr-8.

In one embodiment, the present invention relates to novel systems, methods and compositions for the biocontrol of Potato Virus Y (PVY). In one preferred embodiment, the present invention may include novel genetically modified plant endophytic bacteria, or novel bacterial combinations, which can be used in the biocontrol of PVY through the delivery of heterologous inhibitory RNA molecules.

Another embodiment of the current invention includes the expression and delivery of inhibitory RNAs homologous to the viral genome to efficiently down-regulate or eliminate viral replication and translation of viral proteins of a pathogen or pest. In one preferred embodiment, the inhibitory RNAs may be dsRNA configured to initiate a DICER-mediated RNA interference response in the target plant. In another preferred embodiment, the inhibitory RNAs may be dsRNA, further processed in a bacterial host cell by an engineered RNaseIII configured to generate siRNAs that may be delivered to the target plant and initiate a DICER-independent RNA interference response in the target plant. In another embodiment of the current invention, an engineered RNaseIII configured to generate siRNAs may include novel E. coli RNase III mutant E38A/R107A/R108A (RNase III N3XT™), which in this preferred embodiment may be expressed in planta endophytic bacteria and deliver 22-23 nt interfering RNAs to plants. In this preferred embodiment, the 22-23 nt interfering RNAs may be generated from a co-expressed dsRNA molecules.

Another aim of the current invention includes systems, methods and compositions for the generation of novel botanically compatible plant endophytic bacteria which may be culturable, transformable, and persists only in roots of a plant and that further generate sRNA molecules from co-expressed heterologous dsRNA molecules using RNase III mutants to produce a DICER-independent RNAi response directed to a pathogen or pest in a host plant.

Another aim of the current invention includes systems, methods and compositions for the generation of novel botanically compatible plant endophytic bacteria which may be culturable, transformable, and persists only in roots of a plant and that further generate heterologous dsRNA molecules configured to produce a DICER-dependent RNAi response directed to a pathogen or pest in a host plant.

Another aim of the current invention includes systems, methods and compositions for the generation of novel botanically compatible plant endophytic bacteria which may be culturable, transformable, and persists only in roots of a plant and that further generate sRNA molecules from co-expressed heterologous dsRNA molecules using RNase III mutants to produce a DICER-independent RNAi response directed to a pathogen or pest in a host plant.

Another aim of the current invention includes systems, methods and compositions for the generation of novel botanically compatible plant endophytic bacteria which may be culturable, transformable, and persists only in roots of a plant and that further generate heterologous dsRNA molecules configured to produce a DICER-dependent RNAi response directed to a pathogen or pest in a host plant.

Another embodiment of the current invention includes isolation and identification of novel botanically compatible plant endophytic bacteria which may be culturable, transformable, and persists only in roots of a plant and that further express one or more heterologous inhibitory RNA molecules, such as a double stranded RNA (dsRNA) molecule that may be configured to initiate an RNA interference response in a target plant host. In this preferred embodiment, the genetically engineered plant endophytic bacteria may persist in the plant's roots and express and transport dsRNA molecules that may be configured to initiate an RNA interference response in a target plant. The dsRNA molecules may preferably be hairpin RNA (hpRNA) molecules that may be produced by the endophytic bacteria in the roots and distributed to the rest of the plant through the plant's intracellular architecture.

Another embodiment of the current invention includes methods of against, and/or treating a pathogen or pest in a plant, or seed. In one preferred embodiment, this method may include the isolation and identification of novel botanically compatible plant endophytic bacteria which may be culturable, transformable, and persists only in roots of a plant and that further express one or more heterologous inhibitory RNA molecules, such as a double stranded RNA (dsRNA) molecule that may be configured to initiate an RNA interference response in a target plant host. In this preferred embodiment, the genetically engineered plant endophytic bacteria may persist in the plant's roots and express and transport dsRNA molecules that may be configured to initiate an RNA interference response in a target plant. The dsRNA molecules may preferably be hairpin RNA (hpRNA) molecules that may be produced by the endophytic bacteria in the roots and distributed to the rest of the plant through the plant's intracellular architecture.

Another embodiment of the current invention includes methods of inoculating against, and/or treating a pathogen or pest in a plant, or seed. In one preferred embodiment, this method may include the isolation and identification of novel botanically compatible plant endophytic bacteria which may be culturable, transformable, and persists in a plant and that further express one or more heterologous inhibitory RNA molecules, such as a double stranded RNA (dsRNA) molecule that may be configured to initiate an RNA interference response in a target plant host. In this preferred embodiment, the genetically engineered plant endophytic bacteria may persist in the plant express and transport dsRNA molecules that may be configured to initiate an RNA interference response in a target plant. The dsRNA molecules may preferably be hairpin RNA (hpRNA) molecules that may be produced the endophytic bacteria in, or on, the plant and distributed to the rest of the plant through the plant's intracellular architecture. Another embodiment of the current invention includes the production and isolation of dsRNAs, or siRNAs produced by an engineered RNase enzyme as herein described and administered to a plant that is susceptible to or infected with a pathogen or pest.

In another embodiment, a plant may be inoculated at the root level and remain free of genetically modified organisms throughout the rest of the plant, such as the stems, leaves and fruit. In another embodiment, a plant may be inoculated at the root level and remain free of genetically modified organisms throughout the rest of the plant, such as the stems, leaves and fruit, but the entire plant may be treated or inoculated against a viral pathogen, such as a pathogen or pest, through the distribution of dsRNA molecules throughout the plant. In this manner, the plant may be treated or inoculated against a viral pathogen, such as a pathogen or pest, without requiring the stable genetic transformation and propagation of the plant. This non-GMO strategy may allow the present solution to the treatment of a pathogen or pest to employ a non-GMO embodiment, as well as the ability to rapidly modify one or more dsRNA constructs to adapt to any changes in the target essential genes sequence.

Another embodiment of the current invention includes isolation and identification of novel botanically compatible plant endophytic bacteria that may express one or more heterologous inhibitory RNA molecules, such as a double stranded RNA (dsRNA) molecule that may be configured to initiate an RNA interference response in a target plant host, as well as a heterologous engineered RNaseIII enzyme configured to process the dsRNA molecules into 22-23 nt interfering RNAs in the bacteria that may further be delivered to the tissues of a plant and initiate an RNAi response, and preferably an RNAi response targeting one or more essential genes in a pathogen or pest.

In another embodiment, a plant may be inoculated with a genetically modified endophytic bacteria that expresses a heterologous RNA interference molecule, such as a hpRNA, directed to one or more a pathogen or pest genes, and a heterologous engineered RNaseIII enzyme configured to process the hpRNA molecules into 22-23 nt small interfering RNAs in the bacteria. In this embodiment, the genetically modified endophytic bacteria may be configured to, or naturally be maintained, at the root level such that the remaining tissues of the plant, such as the plant, such as the stems, leaves and fruit, remain free of genetically modified organisms throughout the lifecycle of the plant. In another embodiment, a plant may be inoculated at the root level and there express one or more heterologous RNA interference molecule, such as a hpRNA, directed to one or more a pathogen or pest genes, and a heterologous engineered RNaseIII enzyme configured to process the hpRNA molecules into 22-23 nt siRNAs in the bacteria that may be delivered to the target plant's roots, and distributed through the rest of the plant. In this preferred embodiment, the non-root plant tissues may remain free of genetically modified organisms, such as the stems, leaves and fruit, but the entire plant may be treated or inoculated against a viral pathogen, such as a pathogen or pest, through the production and distribution of siRNA molecules generated by the engineered RNaseIII and delivered throughout the plant from the root tissues. In this manner, the plant may be treated or inoculated against a viral pathogen, such as a pathogen or pest, without requiring the stable genetic transformation and propagation of the plant.

Another embodiment of the current invention includes methods of against, and/or treating a pathogen or pest in a plant, or seed. In one preferred embodiment, this method may include the identification of novel botanically compatible plant endophytic bacteria that may express one or more heterologous inhibitory RNA molecules, such as a double stranded RNA (dsRNA) molecule that may be configured to initiate an RNA interference response in a target plant host, as well as a heterologous engineered RNaseIII enzyme configured to process the dsRNA molecules into 22-23 nt interfering RNAs in the bacteria that may further be delivered to the tissues of a plant and initiate an RNAi response, and preferably an RNAi response targeting one or more essential genes in a pathogen or pest in the plant or seed.

Another embodiment of the current invention includes methods of inoculating against, and/or treating a pathogen or pest in a plant, or seed. In one preferred embodiment, this method may include the inoculating a plant susceptible to, or infected with a pathogen or pest with a genetically modified endophytic bacteria that expresses a heterologous RNA interference molecule, such as a hpRNA, directed to one or more a pathogen or pest genes, and a heterologous engineered RNaseIII enzyme configured to process the hpRNA molecules into 22-23 nt small interfering RNAs in the bacteria that may be delivered to the plant or seed thereby initiating a DICER-independent RNAi response targeting one or more essential genes in a pathogen or pest in the plant or seed. The siRNA molecules may preferably be distributed to the rest of the plant through the plant's intracellular architecture.

In this preferred embodiment, the genetically engineered plant endophytic bacteria may persist in the plant's roots and be vertically and/or horizontally transferred to its progeny or other plants where the genetically engineered plant endophytic bacteria may express and transport dsRNA molecules that may be configured to initiate an RNA interference response in a target plant. The dsRNA molecules may preferably be hairpin RNA (hpRNA) molecules and may further be processed an engineered RNaseIII protein as generally described herein to generate siRNA that may initiate a DICER-independent RNA interference response in a target plant.

In this preferred embodiment, the genetically engineered plant endophytic bacteria may persist in the plant's roots and be vertically and/or horizontally transferred to its progeny or other plants where the genetically engineered plant endophytic bacteria may express and transport dsRNA molecules that may be configured to initiate an RNA interference response in a target plant. The dsRNA molecules may preferably be hairpin RNA (hpRNA) molecules and may further be processed an engineered RNaseIII protein as generally described herein to generate siRNA that may initiate a DICER-independent RNA interference response in a target plant.

Another embodiment of the invention may include isolated polynucleotide sequences encoding an RNA interference molecule, and preferably a hpRNA molecule, directed to a gene of a pathogen or pest.

Another embodiment of the invention may include isolated polynucleotide sequences encoding an RNA interference molecule, and preferably a hpRNA molecule, directed to nucleotide sequence according to SEQ ID NO. 1.

Another embodiment of the invention may include isolated polynucleotide sequences encoding an RNA interference molecule, and preferably a hpRNA molecule, that may be processed to by an engineered RNaseIII enzyme into siRNAs directed to a gene of a pathogen or pest.

Another embodiment of the invention may include isolated polynucleotide sequences encoding an RNA interference molecule, and preferably a hpRNA molecule, that may be processed to by an engineered RNaseIII enzyme into siRNAs directed to nucleotide sequence according to SEQ ID NO. 1.

Another embodiment of the invention may include isolated polynucleotide sequences according to SEQ ID NOs. 2, 4 and 6. Another embodiment of the invention may include one or more of the isolated polynucleotide sequences according to SEQ ID NOs. 2, 4, and 6, operably linked to a promoter, forming an expression vector. Another embodiment of the invention may include one or more bacteria transformed by one or more expression vector configured to express one or more polynucleotide sequences according to SEQ ID NOs. 2, 4, and 6.

Another embodiment of the invention may include isolated polynucleotide sequences according to SEQ ID NO. 2, 4, and 6, and a sequence encoding a heterologous dsRNA. Another embodiment of the invention may include one or more of the isolated polynucleotide sequences according to SEQ ID NO. 2, 4, and 6, and a sequence encoding a heterologous dsRNA operably linked to a promoter forming an expression vector. Another embodiment of the invention may include one or more bacteria transformed by one or more expression vector configured to express one or more polynucleotide sequences according to SEQ ID NO. 2, 4, and 6, and a sequence encoding a heterologous dsRNA. Another embodiment of the invention may include one or more bacteria transformed by one or more expression vector configured to express one or more polypeptide sequences according to amino acid sequences SEQ ID NOs. 3, 5, and 7.

Another embodiment include endophytic bacterial strains Pseudomonas sp. Csr-7 or and Pseudomonas sp. Csr-8. In one preferred embodiment, endophytic bacterial strains Pseudomonas sp. Csr-7, or Pseudomonas sp. Csr-8 may be genetically modified to express one or more polynucleotide sequences according to SEQ ID NO. 2, 4, and 6, and a sequence encoding a heterologous dsRNA, wherein the nucleotide sequences are operably linked to a promoter.

Another embodiment include endophytic bacterial strains Pseudomonas sp. Csr-7, or Pseudomonas sp. Csr-8. In one preferred embodiment, endophytic bacterial strains Pseudomonas sp. Csr-7, or Pseudomonas sp. Csr-8 may be genetically modified to express one or more polynucleotide sequences according to SEQ ID NO. 2, 4, and 6, and a sequence encoding a heterologous dsRNA, wherein the nucleotide sequences are operably linked to a promoter, and wherein endophytic bacterial strains Pseudomonas sp. Csr-7 or and Pseudomonas sp. Csr-8, have been further genetically modified to delete, or disrupt their endogenous or wild type RNaseIII enzymes respectively.

Another embodiment include a plant or seed inoculated with endophytic bacterial strains Pseudomonas sp. Csr-7, or Pseudomonas sp. Csr-8. In one preferred embodiment, the endophytic bacterial strains Pseudomonas sp. Csr-7, or Pseudomonas sp. Csr-8 may be genetically modified to express one or more polynucleotide sequences according to SEQ ID NO. 2, 4, and 6, and a sequence encoding a heterologous dsRNA, wherein the nucleotide sequences are operably linked to a promoter, and wherein endophytic bacterial strains Pseudomonas sp. Csr-7 or and Pseudomonas sp. Csr-8, have been further genetically modified to delete, or disrupt their endogenous or wild type RNaseIII enzymes respectively.

Another embodiment include a pathogen or pest resistant plant, or seed inoculated with endophytic bacterial strains Pseudomonas sp. Csr-7, or Pseudomonas sp. Csr-8. In one preferred embodiment, the endophytic bacterial strains Pseudomonas sp. Csr-7, or Pseudomonas sp. Csr-8 may be genetically modified to express one or more polynucleotide sequences according to SEQ ID NO. 2, 4, and 6, and a sequence encoding a heterologous dsRNA, wherein the nucleotide sequences are operably linked to a promoter, and wherein endophytic bacterial strains Pseudomonas sp. Csr-7 or and Pseudomonas sp. Csr-8, have been further genetically modified to delete, or disrupt their endogenous or wild type RNaseIII enzymes respectively.

Another embodiment include a plant or seed inoculated with endophytic bacterial strains Pseudomonas sp. Csr-7, or Pseudomonas sp. Csr-8. In one preferred embodiment, the endophytic bacterial strains Pseudomonas sp. Csr-7, or Pseudomonas sp. Csr-8 may be genetically modified to express one or more polynucleotide sequences according to SEQ ID NO. 2, 4, and 6, and a sequence encoding a heterologous dsRNA, wherein the nucleotide sequences are operably linked to a promoter, and wherein endophytic bacterial strains Pseudomonas sp. Csr-7 or and Pseudomonas sp. Csr-8, have been further genetically modified to delete, or disrupt their endogenous or wild type RNaseIII enzymes respectively, and which further expresses one or more heterologous helper genes to enhance dsRNA production, stabilization, export and/or delivery Another embodiment may include introducing one or more of the genetically modified endophyte bacteria to a plant, and preferably a plant, through one or more of the methods selected from the group consisting of: through one or more drenching, soaking, spraying, injecting, aerosolized disbursement, environmental aerosolized disbursement, environmental aerosolized disbursement in water sources, lyophilized, freeze-dried, microencapsulated, desiccated, in an aqueous carrier, in a solution, brushing, dressing, dripping, and coating.

As used herein and in the appended claims, the singular forms “a”, “an”, and “the” include plural referents unless the context clearly dictates otherwise. Thus, for example, reference to “a plant” includes a plurality of such plants; reference to “a cell” includes one or more cells and equivalents thereof known to those skilled in the art, and so forth. Similarly, the word “or” is intended to include “and” unless the context clearly indicates otherwise. Hence “comprising A or B” means including A, or B, or A and B. Furthermore, the use of the term “including”, as well as other related forms, such as “includes” and “included”, is not limiting.

The term “about” as used herein is a flexible word with a meaning similar to “approximately” or “nearly”. The term “about” indicates that exactitude is not claimed, but rather a contemplated variation. Thus, as used herein, the term “about” means within 1 or 2 standard deviations from the specifically recited value, or ±a range of up to 20%, up to 15%, up to 10%, up to 5%, or up to 4%, 3%, 2%, or 1% compared to the specifically recited value.

The term “comprising” as used in a claim herein is open-ended and means that the claim must have all the features specifically recited therein, but that there is no bar on additional features that are not recited being present as well. The term “comprising” leaves the claim open for the inclusion of unspecified ingredients even in major amounts. The term “consisting essentially of in a claim means that the invention necessarily includes the listed ingredients and is open to unlisted ingredients that do not materially affect the basic and novel properties of the invention.

A “consisting essentially of claim occupies a middle ground between closed claims that are written in a closed “consisting of format and fully open claims that are drafted in a “comprising format”. These terms can be used interchangeably herein if, and when, this may become necessary. Furthermore, the use of the term “including”, as well as other related forms, such as “includes” and “included”, is not limiting. Notably, where the specification or other parts of this application refer to a polynucleotide sequence, it may also refer to the corresponding protein sequence and vice versa. The term “derived” means an RNase III that was mutated to generate an RNase III mutant.

“RNase III” refers to a naturally occurring enzyme or its recombinant form. The RNase III family of dsRNA-specific endonucleases is characterized by the presence of a highly conserved 9 amino acid stretch in their catalytic center known as the RNaseIII signature motif. Mutants and derivatives are included in the definition. The utility of bacterial RNase III described herein to achieve silencing in mammalian cells further supports the use of RNases from eukaryotes, prokaryotes viruses or archaea in the present embodiments based on the presence of common characteristic consensus sequences. The designations for the mutants are assigned by an amino acid position in a particular RNaseIII isolate. These amino acid positions may vary between RNase III enzymes from different sources. For example, E38 in E. coli corresponds to E37 in Aquifex aeolicus. The positions E38 in E. coli and E37 in A. aeolicus correspond to the first amino acid position of the consensus sequence described above and determined by aligning RNaseIII amino acid sequences from the public databases by their consensus sequences. Embodiments of the invention are not intended to be limited to the actual number designation. Preferred embodiments refer to relative position of the amino acid in the RNaseIII consensus sequence(s). In particular, the invention includes residues 38, 65, 107 and 108 and their corresponding residues across various homologous bacterial RNase III proteins, or homologs.

Mutations in the RNaseIII refer to any of point mutations, additions, deletions (though preferably not in the cleavage domain), and rearrangements (preferably in the domain linking regions). Mutations may be at a single site or at multiple sites in the RNaseIII protein. Mutations can be generated by standard techniques including random mutagenesis, targeted genetics and other methods know by those of ordinary skill in the art.

A further aspect of the invention relates to the use of DNA editing compositions and methods to inhibit, alter, disrupt expression and/or replace one or more target genes, for example through homologous recombination. In various embodiments, one or more target genes may be altered through CRISPR/Cas-9, TALAN or Zinc (Zn2+) finger nuclease systems. In some embodiments, the agent for altering gene expression is CRISPR-Cas9, or a functional equivalent thereof, together with an appropriate RNA molecule arranged to target one or more target genes, such as RNaseIII or any homolog/orthologs thereof. For example, one embodiment of the present invention may include the introduction of one or more guide RNAs (gRNAs) to be utilized by CRISPR/Cas9 system to disrupt, replace, or alter the expression or activity of one or more target genes.

In this context, the gene-editing CRISPR/cas-9 technology is an RNA-guided gene-editing platform that makes use of a bacterially derived protein (Cas9) and a synthetic guide RNA to introduce a double strand break at a specific location within the genome. Editing is achieved by transfecting a cell or a subject with the Cas9 protein along with a specially designed guide RNA (gRNA) that directs the cut through hybridization with its matching genomic sequence. By making use of this technology, it is possible to introduce specific genetic alterations in one or more target genes. In some embodiments, this CRISPR/cas-9 may be utilized to replace one or more existing wild-type genes with a modified version, while additional embodiments may include the addition of genetic elements that alter, reduce, increase or knock-out the expression of a target gene such as endogenous RNaseIII in Csr-7 or Csr-8.

In some embodiments, the agent for altering gene expression is a zinc finger, or zinc finger nuclease or other equivalent. The term “zinc finger nuclease” or “zinc finger nuclease as used herein, refers to a nuclease comprising a nucleic acid cleavage domain conjugated to a binding domain that comprises a zinc finger array. In some embodiments, the cleavage domain is the cleavage domain of the type II restriction endonuclease Fokl. Zinc finger nucleases can be designed to target virtually any desired sequence in a given nucleic acid molecule for cleavage, and the possibility to design zinc finger binding domains to bind unique sites in the context of complex genomes allows for targeted cleavage of a single genomic site in living cells, for example, to achieve a targeted genomic alteration of therapeutic value. Targeting a double-strand break to a desired genomic locus can be used to introduce frame-shift mutations into the coding sequence of a gene due to the error-prone nature of the non-homologous DNA repair pathway. Zinc finger nucleases can be generated to target a site of interest by methods well known to those of skill in the art. For example, zinc finger binding domains with a desired specificity can be designed by combining individual zinc finger motifs of known specificity. The structure of the zinc finger protein Zif268 bound to DNA has informed much of the work in this field and the concept of obtaining zinc fingers for each of the 64 possible base pair triplets and then mixing and matching these modular zinc fingers to design proteins with any desired sequence specificity has been described (Pavletich N P, Pabo Colo. (May 1991). “Zinc finger-DNA recognition: crystal structure of a Zif268-DNA complex at 2.1 A”. Science 252 (5007): 809-17, the entire contents of which are incorporated herein). In some embodiments, separate zinc fingers that each recognizes a 3 base pair DNA sequence are combined to generate 3-, 4-, 5-, or 6-finger arrays that recognize target sites ranging from 9 base pairs to 18 base pairs in length. In some embodiments, longer arrays are contemplated. In other embodiments, 2-finger modules recognizing 6-8 nucleotides are combined to generate 4-, 6-, or 8-zinc finger arrays. In some embodiments, bacterial or phage display is employed to develop a zinc finger domain that recognizes a desired nucleic acid sequence, for example, a desired nuclease target site of 3-30 bp in length.

Zinc finger nucleases, in some embodiments, comprise a zinc finger binding domain and a cleavage domain fused or otherwise conjugated to each other via a linker, for example, a polypeptide linker. The length of the linker determines the distance of the cut from the nucleic acid sequence bound by the zinc finger domain. If a shorter linker is used, the cleavage domain will cut the nucleic acid closer to the bound nucleic acid sequence, while a longer linker will result in a greater distance between the cut and the bound nucleic acid sequence. In some embodiments, the cleavage domain of a zinc finger nuclease has to dimerize in order to cut a bound nucleic acid. In some such embodiments, the dimer is a heterodimer of two monomers, each of which comprise a different zinc finger binding domain. For example, in some embodiments, the dimer may comprise one monomer comprising zinc finger domain A conjugated to a Fokl cleavage domain, and one monomer comprising zinc finger domain B conjugated to a Fokl cleavage domain. In this non-limiting example, zinc finger domain A binds a nucleic acid sequence on one side of the target site, zinc finger domain B binds a nucleic acid sequence on the other side of the target site, and the dimerize Fokl domain cuts the nucleic acid in between the zinc finger domain binding sites.

The term “zinc finger,” as used herein, refers to a small nucleic acid-binding protein structural motif characterized by a fold and the coordination of one or more zinc ions that stabilize the fold. Zinc fingers encompass a wide variety of differing protein structures (see, e.g., Klug A, Rhodes D (1987). “Zinc fingers: a novel protein fold for nucleic acid recognition”. Cold Spring Harb. Symp. Quant. Biol. 52: 473-82, the entire contents of which are incorporated herein by reference). Zinc fingers can be designed to bind a specific sequence of nucleotides, and zinc finger arrays comprising fusions of a series of zinc fingers, can be designed to bind virtually any desired target sequence. Such zinc finger arrays can form a binding domain of a protein, for example, of a nuclease, e.g., if conjugated to a nucleic acid cleavage domain. Different types of zinc finger motifs are known to those of skill in the art, including, but not limited to, Cys2His2, Gag knuckle, Treble clef, Zinc ribbon, Zn2/Cys6, and TAZ2 domain-like motifs (see, e.g., Krishna S, Majumdar I, Grishin N V (January 2003). “Structural classification of zinc fingers: survey and summary”. Nucleic Acids Res. 31 (2): 532-50). Typically, a single zinc finger motif binds 3 or 4 nucleotides of a nucleic acid molecule. Accordingly, a zinc finger domain comprising 2 zinc finger motifs may bind 6-8 nucleotides, a zinc finger domain comprising 3 zinc finger motifs may bind 9-12 nucleotides, a zinc finger domain comprising 4 zinc finger motifs may bind 12-16 nucleotides, and so forth. Any suitable protein engineering technique can be employed to alter the DNA-binding specificity of zinc fingers and/or design novel zinc finger fusions to bind virtually any desired target sequence from 3-30 nucleotides in length (see, e.g., Pabo C O, Peisach E, Grant RA (2001). “Design and selection of novel cys2H is2 Zinc finger proteins”. Annual Review of Biochemistry 70: 313-340; Jamieson A C, Miller J C, Pabo C O (2003). “Drug discovery with engineered zinc-finger proteins”. Nature Reviews Drug Discovery 2 (5): 361-368; and Liu Q, Segal D J, Ghiara J B, Barbas C F (May 1997). “Design of polydactyl zinc-finger proteins for unique addressing within complex genomes”. Proc. Natl. Acad. Sci. U.S.A. 94 (11); the entire contents of each of which are incorporated herein by reference).

Fusions between engineered zinc finger arrays and protein domains that cleave a nucleic acid can be used to generate a “zinc finger nuclease.” A zinc finger nuclease typically comprises a zinc finger domain that binds a specific target site within a nucleic acid molecule, and a nucleic acid cleavage domain that cuts the nucleic acid molecule within or in proximity to the target site bound by the binding domain. Typical engineered zinc finger nucleases comprise a binding domain having between 3 and 6 individual zinc finger motifs and binding target sites ranging from 9 base pairs to 18 base pairs in length. Longer target sites are particularly attractive in situations where it is desired to bind and cleave a target site that is unique in a given genome.

In some embodiments, the agent for altering the target gene is a TALEN system or its equivalent. The term TALEN or “Transcriptional Activator-Like Element Nuclease” or “TALE nuclease” as used herein, refers to an artificial nuclease comprising a transcriptional activator like effector DNA binding domain to a DNA cleavage domain, for example, a Fokl domain. A number of modular assembly schemes for generating engineered TALE constructs have been reported (Zhang, Feng; et. al. (February 2011). “Efficient construction of sequence-specific TAL effectors for modulating mammalian transcription”. Nature Biotechnology 29 (2): 149-53; Geibler, R.; Scholze, H.; Hahn, S.; Streubel, J.; Bonas, U.; Behrens, S. E.; Boch, J. (2011), Shiu, Shin-Han. ed. “Transcriptional Activators of Human Genes with Programmable DNA-Specificity”. PLoS ONE 6 (5): e19509; Cermak, T.; Doyle, E. L.; Christian, M.; Wang, L.; Zhang, Y.; Schmidt, C; Bailer, J. A.; Somia, N. V. et al. (2011). “Efficient design and assembly of custom TALEN and other TAL effector-based constructs for DNA targeting”. Nucleic Acids Research; Morbitzer, R.; Elsaesser, J.; Hausner, J.; Lahaye, T. (2011). “Assembly of custom TALE-type DNA binding domains by modular cloning”. Nucleic Acids Research; Li, T.; Huang, S.; Zhao, X.; Wright, D. A.; Carpenter, S.; Spalding, M. H.; Weeks, D. P.; Yang, B. (2011). “Modularly assembled designer TAL effector nucleases for targeted gene knockout and gene replacement in eukaryotes”. Nucleic Acids Research.; Weber, E.; Gruetzner, R.; Werner, S.; Engler, C; Marillonnet, S. (2011). Bendahmane, Mohammed, ed. “Assembly of Designer TAL Effectors by Golden Gate Cloning”. PLoS ONE 6 (5): e19722; each of which is incorporated herein by reference). Those of skill in the art will understand that TALE nucleases can be engineered to target virtually any genomic sequence with high specificity, and that such engineered nucleases can be used in embodiments of the present technology to manipulate the genome of a cell, e.g., by delivering the respective TALEN via a method or strategy disclosed herein under circumstances suitable for the TALEN to bind and cleave its target sequence within the genome of the cell. In some embodiments, the delivered TALEN targets a gene, such as an endogenous RNaseIII.

In some embodiments, the target gene of a cell, tissue, organ or organism is altered by a nuclease delivered to the cell via a strategy or method disclosed herein, e.g., CRISPR/cas-9, a TALEN, or a zinc-finger nuclease, or a plurality or combination of such nucleases. In some embodiments, a single- or double-strand break is introduced at a specific site within the genome by the nuclease, resulting in a disruption of the target genomic sequence.

In some embodiments, the target genomic sequence is a nucleic acid sequence within the coding region of a target gene. In some embodiments, the strand break introduced by the nuclease leads to a mutation within the target gene that impairs the expression of the encoded gene product.

In some embodiments, a nucleic acid is co-delivered to the cell with the nuclease. In some embodiments, the nucleic acid comprises a sequence that is identical or homologous to a sequence adjacent to the nuclease target site. In some such embodiments, the strand break affected by the nuclease is repaired by the cellular DNA repair machinery to introduce all or part of the co-delivered nucleic acid into the cellular DNA at the break site, resulting in a targeted insertion of the co-delivered nucleic acid, or part thereof. In some embodiments, the insertion results in the disruption or repair of the undesired allele. In some embodiments, the nucleic acid is co-delivered by association to a supercharged protein. In some embodiments, the supercharged protein is also associated to the functional effector protein, e.g., the nuclease. In some embodiments, the delivery of a nuclease to a target cell results in a clinically or therapeutically beneficial alteration of the function of a gene.

In some embodiments, cells from a subject are obtained and a nuclease or other effector protein is delivered to the cells by a system or method provided herein ex vivo. In some embodiments, the treated cells are selected for those cells in which a desired nuclease-mediated genomic editing event has been affected. In some embodiments, treated cells carrying a desired genomic mutation or alteration are returned to the subject they were obtained from.

According to the present invention “sRNA” is small RNA, in particular RNA of a length of 200 nucleotides or less that is not translated into a protein. sRNA may be an RNA molecules digested by one or more of the RNase III mutants described herein. sRNA may include siRNA mRNA, or even dsRNA molecules that may be generated by or initiate an RNAi pathway response which may result in the downregulation of a target gene. “RNAi” refers to gene downregulation or inhibition that is induced by the introduction of a double-stranded RNA molecule.

As used herein, RNA interference (RNAi) is a biological mechanism which leads to post transcriptional gene silencing (PTGS) triggered by double-stranded RNA (dsRNA) molecules, for example provided by hpRNA, to prevent the expression of specific genes. For example, RNA interference may be accomplished as short hpRNA molecules may be imported directly into the cytoplasm, anneal together to form a dsRNA, and then cleaved to short fragments by the DICER enzyme. This enzyme DICER may process the dsRNA into −21-22-nucleotide fragment with a 2-nucleotide overhang at the 3′ end, small interfering RNAs (siRNAs). The antisense strand of siRNA become specific to endonuclease-protein complex, RNA-induced silencing complex (RISC), which then targets the homologous RNA and degrades it at specific site that results in the knock-down of protein expression. Endophytic bacteria that may transmit hpRNA, dsRNA, shRNA, siRNA and microRNA species to plants. In one preferred embodiment, endophytic, entophytic, or any bacteria that may infect or otherwise colonize a plant or live in its surface leaf, stem or root, may be transformed with artificially created genetic constructs, such as plasmids or chromosomal integration, that may generate the inhibitory RNA molecules. In this preferred embodiment, one or more select inhibitory RNA molecules, and preferably hpRNA molecules directed to one or more essential genes of ToBRFV or/and TSWV that, may be expressed in an endophytic bacteria such as Pseudomonas sp. Csr-7 and Csr-8, that further include an engineered E. coli RNase III mutant E38A/R107A/R108A (mc) gene configured to generate siRNAs in a DICER-independent pathway the bacteria was added to the hpRNA expression constructs. In this embodiment, the hpRNA are processed into siRNA molecules that may be delivered to a target, which in a preferred embodiment may be a tomato plant, and down-regulate or eliminate viral replication and translation of viral proteins of ToBRFV or/and TSWV. In further embodiments, the endophytic bacteria such as Pseudomonas sp. Csr-7 and Csr-8 may be localized in the roots of a plant, such that the siRNAs are delivered to the target tomato plant and distributed throughout the plant's intracellular architecture such as the plant's leaves, fruit, and stems. In this manner, the expression of hpRNA, and engineered mc protein, and the production and delivery of siRNAs to the tomato plant may be done without genetically modifying the tomato plant.

Such plasmids may be constructed to be transferrable to other bacteria through conjugation which may allow for widespread environmental inoculation in some instances, as well as vertical transmission among offspring of the plant and any pest or herbivore that ingests the plant matter as an example.

In still other embodiments of the invention, inhibition of the expression of one or more pathogen gene products by RNAi may be obtained through a dsRNA-mediated RNAi action and/or a form of dsRNA known as a hairpin RNA (hpRNA) interference or intron-containing hairpin RNA (ihpRNA) interference. For hpRNA interference, the expression cassette is designed to express an RNA molecule that hybridizes with itself to form a hairpin structure that comprises a single-stranded loop region and a base-paired stem. The base-paired stem region comprises a sense sequence corresponding to all or part of the endogenous messenger RNA encoding the gene product whose expression is to be inhibited, in this case, a pathogen essential gene described herein, and an antisense sequence that is fully or partially complementary to the sense sequence. Alternatively, the base-paired stem region may correspond to a portion of a promoter sequence controlling expression of the gene encoding the target polypeptide to be inhibited. Thus, the base-paired stem region of the molecule generally determines the specificity of the RNA interference. HpRNA molecules are highly efficient at inhibiting the expression of endogenous genes, and the RNA interference they induce is inherited by subsequent generations of plants. See, for example, Chuang and Meyerowitz (2000) Proc. Natl. Acad. Sci. USA 97:4985-4990; Stoutjesdijk et al. (2002) Plant Physiol. 129: 1723-1731; and Waterhouse and Helliwell (2003) Nat. Rev. Genet. 4:29-38. Methods for using hpRNA interference to inhibit or silence the expression of genes are described, for example, in Chuang and Meyerowitz (2000) Proc. Natl. Acad. Sci. USA 97:4985-4990; Stoutjesdijk et al. (2002) Plant Physiol. 129: 1723-1731; Waterhouse and Helliwell (2003) Nat. Rev. Genet. 4:29-38; Pandolfmi et al. BMC Biotechnology 3:7, and U.S. Patent Publication No. 20030175965; each of which is herein incorporated by reference. A transient assay for the efficiency of hpRNA constructs to silence gene expression in vivo has been described by Panstruga et al. (2003) Mol. Biol. Rep. 30:135-140, herein incorporated by reference.

For ihpRNA, the interfering molecules have the same general structure as for hpRNA, but the RNA molecule additionally comprises an intron that is capable of being spliced in the cell in which the ihpRNA is expressed. The use of an intron minimizes the size of the loop in the hairpin RNA molecule following splicing, and this increases the efficiency of interference. See, for example, Smith et al. (2000) Nature 407:319-320. In fact, Smith et al. show 100% suppression of endogenous gene expression using ihpRNA-mediated interference. Methods for using ihpRNA interference to inhibit the expression of endogenous plant genes are described, for example, in Smith et al. (2000) Nature 407:319-320; Wesley et al. (2001) Plant J 27:581-590; Wang and Waterhouse (2001) Curr. Opin. Plant Biol. 5: 146-150; Waterhouse and Helliwell (2003) Nat. Rev. Genet. 4:29-38; Helliwell and Waterhouse (2003) Methods 30:289-295, and U.S. Patent Publication No. 20030180945, each of which is herein incorporated by reference.

The term “gene” or “gene sequence” refers to a coding region operably joined to appropriate regulatory sequences capable of regulating the expression of the gene product (e.g., a polypeptide or a functional RNA) in some manner. A gene includes untranslated regulatory regions of DNA (e.g., promoters, enhancers, repressors, etc.) preceding (up-stream) and following (down-stream) the coding region (open reading frame, ORF) as well as, where applicable, intervening sequences (e.g., introns) between individual coding regions (e.g., exons). The term “structural gene” as used herein is intended to mean a DNA sequence that is transcribed into mRNA which is then translated into a sequence of amino acids characteristic of a specific polypeptide.

As used herein, “inhibit, “inhibition,” “suppress,” “downregulate” or “silencing” refers to partial or complete loss-of-function through targeted inhibition of gene expression in a cell and may also be referred to as “knock down,” preferably through an RNAi pathway response.

Depending on the circumstances and the biological problem to be addressed, it may be preferable to partially reduce gene expression. Alternatively, it might be desirable to reduce gene expression as much as possible. The extent of silencing may be determined by any method known in the art, some of which are summarized in International Publication No. WO 99/32619, incorporated herein by reference. As used herein, ““inhibit, “inhibition,” ““suppress,” “downregulate,” or “silencing” of the level or activity of an agent, such as, for example, a preRNA, mRNA, rRNA, tRNA, snoRNA, snRNA expressed by the target gene, and/or of the protein product encoded by it, means that the amount is reduced by 10% or more, for example, 20% or more, preferably 30% or more, more preferably 50% or more, even more preferably 70% or more, most preferably 80% or more, for example, 90%, relative to a cell or organism lacking a dsRNA molecule of the disclosure.

“Large double-stranded RNA” refers to any dsRNA or hairpin having a double-stranded region greater than about 40 base pairs (bp) for example, larger than 100 bp, or more, particularly larger than 300 bp. The sequence of a large dsRNA may represent one or more segments of one or more mRNAs or the entire mRNAs. The maximum size of the large dsRNA is not limited herein. The dsRNA may include modified bases where the modification may be to the phosphate sugar backbone or to the nucleotide. Such modifications may include a nitrogen or sulfur heteroatom, or any other modification known in the art. The dsRNA may be made enzymatically, by recombinant techniques, and/or by chemical synthesis or using commercial kits such as MEGASCRIPT® (Ambion, Austin, Tex.) and methods known in the art. An embodiment of the invention utilizes HiScribe™ (New England Biolabs, Inc., Beverly, Mass.) for making large dsRNA. Other methods for making and storing large dsRNA are described in International Publication No. WO 99/32619. The double-stranded structure may be formed by a self-complementary RNA strand such as occurs for a hairpin or a micro RNA, or by annealing of two distinct complementary RNA strands. As used herein a “wild type” means a cell or organism that does not contain the heterologous recombinant DNA that expressed a protein or element that imparts an enhanced trait as described herein.

“Expression” or “expressing” refers to production of a functional product, such as, the generation of an RNA transcript from an introduced construct, an endogenous DNA sequence, or a stably incorporated heterologous DNA sequence. A nucleotide encoding sequence may comprise intervening sequence (e.g., intrans) or may lack such intervening non-translated sequences (e.g., as in cDNA). Expressed genes include those that are transcribed into mRNA and then translated into protein and those that are transcribed into RNA but not translated (for example, siRNA, transfer RNA, and ribosomal RNA). The term may also refer to a polypeptide produced from an mRNA generated from any of the above DNA precursors. Thus, expression of a nucleic acid fragment, such as a gene or a promoter region of a gene, may refer to transcription of the nucleic acid fragment (e.g., transcription resulting in mRNA or other functional RNA) and/or translation of RNA into a precursor or mature protein (polypeptide), or both.

An “expression cassette or “expression vector” or “vector” refers to a nucleic acid construct, which when introduced into a host cell, results in transcription and/or translation of an RNA or polypeptide, respectively. More specifically, the term “vector” refers to some means by which DNA, RNA, a protein, or polypeptide can be introduced into a host. The polynucleotides, protein, and polypeptide which are to be introduced into a host can be therapeutic or prophylactic in nature; can encode or be an antigen; can be regulatory in nature, etc. There are various types of vectors including virus, plasmid, bacteriophages, cosmids, and bacteria. Again, more specifically, “expression vector” is nucleic acid capable of replicating in a selected host cell or organism. An expression vector can replicate as an autonomous structure, or alternatively can integrate, in whole or in part, into the host cell chromosomes or the nucleic acids of an organelle, or it is used as a shuttle for delivering foreign DNA to cells, and thus replicate along with the host cell genome. Thus, an expression vector are polynucleotides capable of replicating in a selected host cell, organelle, or organism, e.g., a plasmid, virus, artificial chromosome, nucleic acid fragment, and for which certain genes on the expression vector (including genes of interest) are transcribed and translated into a polypeptide or protein within the cell, organelle or organism; or any suitable construct known in the art, which comprises an “expression cassette.” In contrast, as described in the examples herein, a “cassette” is a polynucleotide containing a section of an expression vector of this invention. The use of the cassettes assists in the assembly of the expression vectors. An expression vector is a replicon, such as plasmid, phage, virus, chimeric virus, or cosmid, and which contains the desired polynucleotide sequence operably linked to the expression control sequence(s). A polynucleotide sequence is operably linked to an expression control sequence(s) (e.g., a promoter and, optionally, an enhancer) when the expression control sequence controls and regulates the transcription and/or translation of that polynucleotide sequence.

The term “genome” encompasses not only chromosomal DNA found within the nucleus, but organelle DNA found within subcellular components (e.g., mitochondrial, plastid) of the cell. As used herein, the term “genome” refers to the nuclear genome unless indicated otherwise.

As used herein, the term “eukaryotic-like RNA” or “eukaryotic-like mRNA” refers to an RNA molecule expressed in a prokaryotic or other non-eukaryotic systems that is competent to be expressed in a recipient eukaryotic cell.

Notably, all DNA sequences provided may encompass all RNA and amino acid sequences, and vice versa as would be ascertainable by those of ordinary skill in the art, for example through Uracil substitutions as well as redundant codons. Additionally, all sequences include codon-optimized embodiments as would be ascertainable by those of ordinary skill in the art. As such, the term “encoding” or “coding sequence” or “coding” means both encoding a nucleotide and/or amino acid sequence and vice versa.

The term “heterologous” refers to a nucleic acid fragment or protein that is foreign to its surroundings. In the context of a nucleic acid fragment, this is typically accomplished by introducing such fragment, derived from one source, into a different host. Heterologous nucleic acid fragments, such as coding sequences that have been inserted into a host organism, are not normally found in the genetic complement of the host organism. As used herein, the term “heterologous” also refers to a nucleic acid fragment derived from the same organism, but which is located in a different, e.g., non-native, location within the genome of this organism. Thus, the organism can have more than the usual number of copy(ies) of such fragment located in its(their) normal position within the genome and in addition, in the case of plant cells, within different genomes within a cell, for example in the nuclear genome and within a plastid or mitochondrial genome as well. A nucleic acid fragment that is heterologous with respect to an organism into which it has been inserted or transferred is sometimes referred to as a “transgene.”

“Host cell” means a cell which contains an expression vector and supports the replication and/or expression of that vector. The term “introduced” means providing a nucleic acid (e.g., an expression construct) or protein into a cell. “Introduced” includes reference to the incorporation of a nucleic acid into a eukaryotic or prokaryotic cell where the nucleic acid may be incorporated into the genome of the cell and includes reference to the transient provision of a nucleic acid or protein to the cell. “Introduced” includes reference to stable or transient transformation methods, as well as sexually crossing. Thus, “introduced” in the context of inserting a nucleic acid fragment (e.g., a recombinant DNA construct/expression construct) into a cell, can mean “transfection” or “transformation” or “transduction”, and includes reference to the incorporation of a nucleic acid fragment into a eukaryotic or prokaryotic cell where the nucleic acid fragment may be incorporated into the genome of the cell (e.g., chromosome, plasmid, plastid, or mitochondrial DNA), converted into an autonomous replicon, or transiently expressed (e.g., transfected mRNA).

As used herein, “nucleic acid” or “nucleotide sequence” means a polynucleotide (or oligonucleotide), including single or double-stranded polymers of deoxyribonucleotides or ribonucleotide bases, and unless otherwise indicated, encompasses naturally occurring and synthetic nucleotide analogues having the essential nature of natural nucleotides in that they hybridize to complementary single stranded nucleic acids in a manner similar to naturally occurring nucleotides. Nucleic acids may also include fragments and modified nucleotide sequences. Nucleic acids disclosed herein can either be naturally occurring, for example genomic nucleic acids, or isolated, purified, nongenomic nucleic acids, including synthetically produced nucleic acid sequences such as those made by solid phase chemical oligonucleotide synthesis, enzymatic synthesis, or by recombinant methods, including for example, cDNA, codon-optimized sequences for efficient expression in different transgenic plants reflecting the pattern of codon usage in such plants, nucleotide sequences that differ from the nucleotide sequences disclosed herein due to the degeneracy of the genetic code but that still encode the protein(s) of interest disclosed herein, nucleotide sequences encoding the presently disclosed protein(s) comprising conservative (or non-conservative) amino acid substitutions that do not adversely affect their normal activity, PCR-amplified nucleotide sequences, and other non-genomic forms of nucleotide sequences familiar to those of ordinary skill in the art.

The term “sequence identity” or “identity,” as used herein in the context of two nucleic acid or polypeptide sequences, refers to the residues in the two sequences that are the same when aligned for maximum correspondence over a specified comparison window.

As used herein, the term “percentage of sequence identity” may refer to the value determined by comparing two optimally aligned sequences (e.g., nucleic acid sequences) over a comparison window, wherein the portion of the sequence in the comparison window may comprise additions or deletions (e.g., gaps) as compared to the reference sequence (which does not comprise additions or deletions) for optimal alignment of the two sequences. The percentage is calculated by determining the number of positions at which the identical nucleotide or amino acid residue occurs in both sequences to yield the number of matched positions, dividing the number of matched positions by the total number of positions in the comparison window, and multiplying the result by 100 to yield the percentage of sequence identity. A sequence that is identical at every position in comparison to a reference sequence is said to be 100% identical to the reference sequence, and vice-versa. Methods for aligning sequences for comparison are well-known in the art. Various programs and alignment algorithms are described in, for example: Smith and Waterman (1981) Adv. Appl. Math. 2:482; Needleman and Wunsch (1970) J. Mol. Biol. 48:443; Pearson and Lipman (1988) Proc. Natl. Acad. Sci. U.S.A. 85:2444; Higgins and Sharp (1988) Gene 73:237-44; Higgins and Sharp (1989) CABIOS 5:151-3; Corpet et al. (1988) Nucleic Acids Res. 16:10881-90; Huang et al. (1992) Comp. Appl. Biosci. 8:155-65; Pearson et al. (1994) Methods Mol. Biol. 24:307-31; Tatiana et al. (1999) FEMS Microbiol. Lett. 174:247-50. A detailed consideration of sequence alignment methods and homology calculations can be found in, e.g., Altschul et al. (1990) J. Mol. Biol. 215:403-10. The National Center for Biotechnology information (NCBI) Basic Local Alignment Search Tool (BLAST™; Altschul et al. (1990)) is available from several sources, including the National Center for Biotechnology Information (Bethesda, Md.), and on the internet, for use in connection with several sequence analysis programs. A description of how to determine sequence identity using this program is available on the internet under the “help” section for BLAST™. For comparisons of nucleic acid sequences, the “Blast 2 sequences” function of the BLAST™ (Blastn) program may be employed using the default BLOSUM62 matrix set to default parameters. Nucleic acid sequences with even greater similarity to the reference sequences will show increasing percentage identity when assessed by this method.

“Nucleic acid construct” or “construct” refers to an isolated polynucleotide which can be introduced into a host cell, for example a plasmid. This construct may comprise any combination of deoxyribonucleotides, ribonucleotides, and/or modified nucleotides. This construct may comprise an expression cassette that can be introduced into and expressed in a host cell.

“Operably linked” refers to a functional arrangement of elements. A first nucleic acid sequence is operably linked with a second nucleic acid sequence when the first nucleic acid sequence is placed in a functional relationship with the second nucleic acid sequence. For instance, a promoter is operably linked to a coding sequence if the promoter effects the transcription or expression of the coding sequence. The control elements need not be contiguous with the coding sequence, so long as they function to direct the expression thereof. Thus, for example, intervening untranslated yet transcribed sequences can be present between a promoter and the coding sequence and the promoter can still be considered “operably linked” to the coding sequence.

The terms “peptide”, “polypeptide”, and “protein” are used to refer to polymers of amino acid residues. These terms are specifically intended to cover naturally occurring biomolecules, as well as those that are recombinantly or synthetically produced, for example by solid phase synthesis.

The term “promoter” or “regulatory element” refers to a region or nucleic acid sequence located upstream or downstream from the start of transcription and which is involved in recognition and binding of RNA polymerase and/or other proteins to initiate transcription of RNA. Promoters need not be of plant or algal origin. For example, promoters derived from plant viruses, such as the CaMV35S promoter, or from other organisms, can be used in variations of the embodiments discussed herein. Promoters useful in the present methods include, for example, constitutive, strong, weak, tissue-specific, cell-type specific, seed-specific, inducible, repressible, and developmentally regulated promoters. Examples of suitable promoters for gene suppressing cassettes include, but are not limited to, T7 promoter, bla promotor, U6 promoter, pol II promoter, Ell promoter, and CMV promoter and the like.

A “cell type-specific” promoter primarily drives expression in certain cell types in one or more organs. An “inducible” promoter may be a promoter which may be under environmental control or induced by a secondary molecule or compound Tissue-specific, tissue-preferred, cell type specific, and inducible promoters constitute the class of “non-constitutive” promoters. A “constitutive” promoter is a promoter which may be active under most environmental conditions or in most cell or tissue types.

As used herein, the term “transformation” or “genetically modified” refers to the transfer of one or more nucleic acid molecule(s) into a cell. A microorganism is “transformed” or “genetically modified” by a nucleic acid molecule transduced into the bacteria when the nucleic acid molecule becomes stably replicated by the bacteria. As used herein, the term “transformation” or “genetically modified” encompasses all techniques by which a nucleic acid molecule can be introduced into, such as a bacterium.

As used herein, a “genetically modified plant or “transgenic plant” is one whose genome has been altered by the incorporation of exogenous genetic material, e.g. by transformation as described herein. The term “transgenic plant” is used to refer to the plant produced from an original transformation event, or progeny from later generations or crosses of a transgenic plant so long as the progeny contains the exogenous genetic material in its genome. By “exogenous” is meant that a nucleic acid molecule, for example, a recombinant DNA, originates from outside the plant into which it is introduced. An exogenous nucleic acid molecule may comprise naturally or non-naturally occurring DNA and may be derived from the same or a different plant species than that into which it is introduced.

“Stable transformation” is intended to mean that the nucleotide construct introduced into a host and integrates into the genome of the plant and is capable of being inherited by the progeny thereof. The nucleic acid molecule can be transiently expressed or non-stably maintained in a functional form in the cell for less than three months e.g. is transiently expressed.

The terms “plant” or “plants” that can be used in the present methods broadly include the classes of higher and lower plants amenable to transformation techniques, including angiosperms (monocotyledonous and dicotyledonous plants), Gymnosperms, ferns, and unicellular and multicellular algae. The term “plant” also includes plants which have been modified by breeding, mutagenesis, or genetic engineering (transgenic and non-transgenic plants). It includes plants of a variety of ploidy levels, including aneuploid, polyploid, diploid, haploid, and hemizygous. The plant may be in any form including suspension cultures, embryos, meristematic regions, callus tissue, gametophytes, sporophytes, pollen, microspores, whole plants, shoot vegetative organs/structures (e.g. leaves, stems and tubers), roots, flowers and floral organs/structures, seed (including embryo, endosperm, and seed coat) and fruit, plant tissue (e.g. vascular tissue, ground tissue, and the like) and cells, and progeny of same.

In one embodiment, the plant is a monocot, or a cell of a monocot plant. For example, the monocot plant is in the gramineae and cereal groups. Non-limiting exemplary monocot species include grains, tropical fruits and flowers, bananas, maize, rice, barley, duckweed, gladiolus, sugar cane, pineapples, dates, onions, rice, sorghum, turfgrass and wheat. In another embodiment, the plant is a dicot, or a cell of a monocot plant. For example, the dicot plant is selected from the group consisting of Anacardiaceae (e.g., cashews, pistachios), Asteraceae (e.g., asters and all the other composite flowers), Brassicaceae (e.g., cabbage, turnip, and other mustards), Cactaceae (e.g., cacti), Cucurbitaceae (e.g., watermelon, squashes), Euphorbiaceae (e.g., cassaya (manioc)), Fabaceae (e.g., beans and all the other legumes), Fagaceae (e.g., oaks), Geraniales (e.g., geraniums), Juglandaceae (e.g., pecans), Linaceae (e.g., flax), Malvaceae (e.g., cotton), Oleaceae (e.g., olives, ashes, lilacs), Rosaceae (e.g., roses, apples, peaches, strawberries, almonds), Rubiaceae (e.g., coffee), Rutaceae (e.g., oranges and other citrus fruits), Solanaceae (e.g., potatoes, tomatoes, tobacco), Theaceae (e.g., tea), and Vitaceae (e.g., grapes). The term “plant” or “target plant” includes any plant sustainable to a pathogen. It further includes whole plants, plant organs, progeny of whole plants or plant organs, embryos, somatic embryos, embryo-like structures, protocorms, protocorm-like bodies (PLBs), and suspensions of plant cells. Plant organs comprise, e.g., shoot vegetative organs/structures (e.g., leaves, stems and tubers), roots, flowers and floral organs/structures (e.g., bracts, sepals, petals, stamens, carpels, anthers and ovules), seed (including embryo, endosperm, and seed coat) and fruit (the mature ovary), plant tissue (e.g., vascular tissue, ground tissue, and the like) and cells (e.g., guard cells, egg cells, trichomes and the like). The class of plants that can be used in the method of the invention is generally as broad as the class of higher and lower plants amenable to the molecular biology and plant breeding techniques described herein, specifically angiosperms (monocotyledonous (monocots) and dicotyledonous (dicots) plants including eudicots. It includes plants of a variety of ploidy levels, including aneuploid, polyploid, diploid, haploid and hemizygous. The genetically altered plants described herein can be monocot crops, such as, sorghum, maize, wheat, rice, barley, oats, rye, millet, and triticale. The invention may also include Cannabaceae and other Cannabis strains, such as C. sativa generally. Examples of additional plants species of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum [Sorghum bicolor, Sorghum vulgare), millet (e.g., pearl millet (Pennisetum glaucum), proso millet (Panicum miliaceum), foxtail millet (Setaria italica), finger millet (Eleusine coracana), sunflower (Helianthus annuus), safflower (Carthamus tinctorius), wheat (Triticum aestivum), soybean (Glycine max), tobacco (Nicotiana tabacum), potato (Solarium tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolid), almond (Prunus amygdalus), sugar beets (Beta vulgaris), sugarcane (Saccharum spp.), oats, barley; vegetables, ornamentals, and conifers. Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis, such as cucumbers (C. sativus), cantaloupe (C. cantalupensis), and musk melon (C. melo). Ornamentals include azalea (Rhododendron spp), hydrangea (Macrophylla hydrangea), hibiscus (Hibiscus rosasanensis), roses (Rosa spp.), tulips (Tulipa spp.), daffodils (Narcissus spp), petunias (Petunia hybrida), carnations (Dianthus caryophyllus), poinsettias (Euphorbia pukherrima), and chrysanthemums. Conifers that may be employed in practicing the present invention include, for example, pines such as loblolly pine (Finns taeda), slash pine (Pinus elUoiii), ponderosa pine (Pinus ponderosa), lodgepole pine (Finns contorta), and Monterey pine (Pinus radiata), Douglas-fir (Pseudotsuga menziesii), Western hemlock (Tsuga canadensis), Sitka spruce (Picea glauca), redwood (Sequoia sempervirens), true firs such as silver fir (Abies amabilis) and balsam fir (Abies balsamea), and cedars such as Western red cedar (Thuja plicatd) and Alaska yellow-cedar (Chamaecyparis nootkatensis). In specific embodiments, plants of the present invention are crop plants (for example, as noted above, corn, alfalfa, sunflower, Brassica, soybean, cotton, safflower, peanut, sorghum, wheat, millet, tobacco, etc.).

Any gene being expressed in a cell (preferably a plant cell) can be targeted. A gene that is expressed in the cell is one that is transcribed to yield RNA (e.g., miRNA) and, optionally, a protein. The target gene can be an endogenous gene or an exogenous or foreign gene (i.e., a transgene or a pathogen gene). For example, a transgene that is present in the genome of a cell as a result of genomic integration of the viral delivery construct can be regulated using inhibitory RNA according to the invention. The foreign gene can be integrated into the host genome (preferably the chromosomal DNA), or it may be present on an extra-chromosomal genetic construct such as a plasmid or a cosmid. For example, the target gene may be present in the genome of the cell into which the interfering RNA is introduced through the novel trans-kingdom method described herein, or similarly in the genome of a pathogen, such as a virus, a bacterium, a fungus or a protozoan, which is capable of infecting such organism or cell.

Preferably the target gene is an endogenous gene of the cell or a heterologous gene relative to the genome of the cell, such as a pathogen gene. Preferably, the gene of a pathogen is from a pathogen capable of infecting a eukaryotic organism. Most preferably, said pathogen is selected from the group of virus, bacteria, fungi and nematodes as noted above. By expressing the inhibitory RNA of the invention in plants, not only plant genes can function as target genes for gene silencing, but also genes of organisms which infect plants or eat plants (as food or feed). Thus, the target gene can also be a gene of an animal or plant pathogen. The target gene is preferably selected from the group consisting of genes in a plant or of a plant infecting pathogen. Preferably, the expression of the target gene (as measured by the expressed RNA or protein) is reduced, inhibited or attenuated by at least 10%, preferably at least 30% or 40%, preferably at least 50% or 60%, more preferably at least 80%, most preferably at least 90% or 95% or 100%.

The levels of target products such as transcripts or proteins may be decreased throughout an organism such as a plant, pest or herbivore, or such decrease in target products may be localized in one or more specific organs or tissues of the organism. For example, the levels of products may be decreased in one or more of the tissues and organs of a plant including without limitation: roots, tubers, stems, leaves, stalks, fruit, berries, nuts, bark, pods, seeds and flowers. A preferred organ is a seed of a plant. A broad variety of target genes can be modulated by using the method of the invention, including genes in a plant but also genes or plant infecting or eating pathogens, animals, or even human. Preferably, the target gene is selected from the group consisting of plant endogenous, transgenes, or genes from a plant infecting pathogen. More preferably the plant infecting pathogen is selected from the group consisting of viruses, fungi, bacteria, insects, and nematodes.

In the case of pathogens, the target or essential gene may, for example, be a housekeeping or other gene, which is essential for viability or proliferation of the pathogen. The attenuation or silencing of the target gene may have various effects (also depending on the nature of the target gene). Preferably, silencing or attenuating said target gene results in loss or reduction or the pathogen's harmful effects, i.e., pathogenicity, or an agronomic trait. Said agronomic trait may preferably be selected from the group consisting of disease resistance, herbicide resistance, resistance against biotic or abiotic stress, and improved nutritional value. In this context, the target gene may, for example, be preferably selected from the group consisting of genes involved in the synthesis and/or degradation of proteins, peptides, fatty acids, lipids, waxes, oils, starches, sugars, carbohydrates, flavors, odors, toxins, carotenoids, hormones, polymers, flavinoids, storage proteins, phenolic acids, alkaloids, lignins, tannins, celluloses, glycoproteins, and glyco lipids. All these sequences are well known to the person skilled in the art and can be easily obtained from DNA data bases by those of ordinary skill in the art (e.g., GenBank).

In certain embodiments, the novel trans-kingdom delivery of inhibitory RNA molecules, namely the methods and means of the invention may be especially suited for obtaining pathogen (e.g., virus or nematode) resistance, in eukaryotic cells or organisms, particularly in plant cells and plants. It is expected that the inhibitory RNA molecules (or the dsRNA molecules derived therefrom) produced by transcription in a host organism (e.g., a plant), can spread systemically throughout the organism. Thus, it is possible to reduce the phenotypic expression of a nucleic acid in cells of a non-transgenic scion of a plant grafted onto a transgenic stock comprising the genes of the invention (or vice versa) a method which may be important in horticulture, viticulture or in fruit production. A resistance to plant pathogens such as arachnids, fungi, insects, nematodes, protozoans, viruses, bacteria and diseases can be achieved by reducing the gene expression of genes which are essential for the growth, survival, certain developmental stages (for example pupation), or the multiplication of a certain pathogen. A suitable reduction can bring about a complete inhibition of the above steps, but also a delay of one or more steps. This may include plant genes which, for example, allow the pathogen to enter, but may also be pathogen-homologous genes. Preferably, the inhibitory RNA (such as hpRNA or the dsRNA derived therefrom) is directed against genes of the pathogen. For example, plants can be treated with suitable formulations of above mentioned agents, for example sprayed or dusted; the plants themselves, however, may also comprise the agents in the form of a transgenic organism and pass them on to the pathogens, for example in the form of a stomach poison. Various essential genes of a variety of pathogens are known to those of ordinary skill in the art (for example for nematode resistance: WO 93/10251, WO 94/17194). Another aspect of methods of the novel trans-kingdom delivery of inhibitory RNA molecules described here provides a method where the target gene for suppression encodes a protein in a plant pathogen (e.g., an insect or nematode). In this aspect, a method comprises introducing into the genome of a pathogen-targeted plant a nucleic acid construct comprising DNA, such as a plasmid, which is transcribed into a inhibitory RNA, such as a hpRNA, that forms at least one dsRNA molecule which is effective for reducing expression of a target gene within the pathogen when the pathogen (e.g., insect or nematode) ingests or infects cells from said plant. In a preferred embodiment, the gene suppression is fatal to the pathogen. Most preferred as a pathogen are fungal pathogens, to the extent not already listed elsewhere, such as Phytophthora infestans, Fusarium nivale, Fusarium graminearum, Fusarium culmorum, Fusarium oxysporum, Blumeria graminis, Magnaporthe grisea, Sclerotinia sclerotium, Septoria nodorum, Septoria tritici, Alternaria brassicae, Phoma lingam, and nematodes such as Globodera rostochiensis, G. pallida, Heterodera schachtii, Heterodera avenae, Ditylenchus dipsaci, Anguma tritici and Meloidogyne hapla.

Resistance to pathogenic viruses can be obtained for example by reducing the expression of a viral coat protein, a viral replicase, a viral protease, a, a structural protein, a toxin and the like. A large number of plant viruses, and suitable target genes are known to those of ordinary skill in the art. The methods and compositions of the present invention are especially useful to obtain nematode resistant plants (for target genes see e.g., WO 92/21757, WO 93/10251, WO 94/17194).

A “plant pathogen” or “pathogen” refers to an organism (bacteria, virus, protist, algae or fungi) that infects plants or plant components. Examples include molds, fungi and rot that typically use spores to infect plants or plant components (e.g. fruits, vegetables, grains, stems, roots). A “plant pathogen” also includes all genes necessary for the pathogenicity or pathogenic effects in the plant, or that by their suppression or elimination, such effects are reduced or eliminated.

In one preferred embodiment, the present invention may be applied to one or more of the following non-limiting group of plant viruses, including pathogen gene targets, generally referred to as gene targets, or essential genes, which would be recognized and available to those of ordinary skill in the art without undue experimentation identified in Sayre et al, PCT/US2017/064977, at 26-30, being incorporated herein by reference)

In one embodiment, a donor endophyte may be engineered to synthesize and/or deliver eukaryotic-like mRNAs that are engineered to produce proteins that are configured to generate a phenotypic, biochemical, metabolic, or other directed modulations in a recipient eukaryotic organism. For example, in one preferred embodiment, a donor prokaryotic organi sin may be engineered to synthesize and deliver eukaryotic-like mRNAs, or mRNAs to a eukaryotic host that, when translated, may induce a new phenotype. In one embodiment, such a phenotypic change may include increases in one or more metabolic or other growth pathways. Additional phenotypic changes may include physical, and/or biochemical changes not previously present in the wild-type host. Additional phenotypic changes may include enhanced, or even new, metabolic processes, or even the production of a non-naturally occurring compounds or other molecules of interest. Examples of such compounds and molecules, of interest may include vaccine or other disease resistant molecules that may provide enhanced pathogen resistance in the eukaryotic host. Additional examples may include the production of one or more toxins or other compounds that may be lethal to a specific pathogen, insect, or other pest.

The term “prokaryotic” is meant to include all bacteria, archaea, and/or cyanobacteria which can be transformed or transfected with a nucleic acid and express a eukaryotic-like RNA of the invention. Prokaryotic hosts may include gram negative as well as gram positive bacteria. The term “eukaryotic” is meant to include yeast, algae, plants, higher plants, insect, and mammalian cells.

“Target” or “essential gene” refers to any gene or mRNA of interest. Indeed, any of the genes previously identified by genetics or by sequencing may represent a target. Target genes or mRNA may include developmental genes and regulatory genes as well as metabolic or structural genes or genes encoding enzymes. The target gene may be expressed in those cells in which a phenotype is being investigated or in an organism in a manner that directly or indirectly impacts a phenotypic characteristic. The target gene may be endogenous or exogenous. An “essential gene,” for example may be a gene necessary for survival, replication or pathogenicity in a pathogen. Such cells include any cell in the body of an adult or embryonic animal or plant including gamete or any isolated cell such as occurs in an immortal cell line or primary cell culture.

As used herein “resistance” or “improved resistance” in a plant to disease conditions is an indication that the plant is more able to reduce disease burden than a non-resistant or less resistant plant. Resistance is a relative term, indicating that a “resistant” plant is more able to reduce disease burden compared to a different (less resistant) plant (e.g., a different plant variety) grown in similar disease conditions. One of skill will appreciate that plant resistance to disease conditions varies widely and can represent a spectrum of more-resistant or less-resistant phenotypes. However, by simple observation, one of skill can generally determine the relative resistance of different plants, plant varieties, or plant families under disease conditions, and furthermore, will also recognize the phenotypic gradations of “resistant.”

Unless otherwise stated, nucleic acid sequences in the text of this specification are given, when read from left to right, in the 5′ to 3′ direction. Nucleic acid sequences may be provided as DNA or as RNA, as specified; disclosure of one necessarily defines the other, as is known to one of ordinary skill in the art and is understood as included in embodiments where it would be appropriate. Nucleotides may be referred to by their commonly accepted single-letter codes.

Unless otherwise indicated, amino acid sequences are written left to right in amino to carboxyl orientation, respectively. Amino acids may be referred to herein by either their commonly known three letter symbols or by the one-letter symbols as generally understood by those skilled in the relevant art.

Regarding disclosed ranges, the endpoints of all ranges directed to the same component or property are inclusive and independently combinable (e.g., ranges of “about 25%, or, more, about 5% to about 20 wt. %,” is inclusive of the endpoints and all intermediate values of the ranges of “about 5% to about 25%,” etc.). Numeric ranges recited with the specification are inclusive of the numbers defining the range and include each integer within the defined range. Notably, all peptides disclosed in specifically encompass peptides having conservative amino acid substitutions. As used herein, “conservative amino acid substitutions” means the manifestation that certain amino acids can be substituted for other amino acids in a protein structure without appreciable loss of biochemical or biological activity. Since it is the interactive capacity and nature of a protein that defines that protein's biological functional activity, certain amino acid sequence substitutions can be made in a protein sequence, and, of course, the underlying DNA coding sequence, and nevertheless obtain a protein with like properties. Thus, various changes can be made in the amino acid sequences disclosed herein, or in the corresponding DNA sequences that encode these amino acid sequences, without appreciable loss of their biological utility or activity.

Examples of amino acid groups defined in this manner include: a “charged polar group,” consisting of glutamic acid (Glu), aspartic acid (Asp), asparagine (Asn), glutamine (Gln), lysine (Lys), arginine (Arg) and histidine (His); an “aromatic, or cyclic group,” consisting of proline (Pro), phenylalanine (Phe), tyrosine (Tyr) and tryptophan (Trp); and an “aliphatic group” consisting of glycine (Gly), alanine (Ala), valine (Val), leucine (Leu), isoleucine (Ile), methionine (Met), serine (Ser), threonine (Thr) and cysteine (Cys).

In a further embodiment, a composition including a genetically modified bacteria configured to express one or more RNase III mutants that produce sRNA may be formulated as feed, such as a plant feed, and/or a water dispersible granule or powder that may further be configured to be dispersed into the environment. In yet a further embodiment, the compositions of the present invention may also comprise a wettable powder, spray, emulsion, colloid, aqueous or organic solution, dust, pellet, or colloidal concentrate. Dry forms of the compositions may be formulated to dissolve immediately upon wetting, or alternatively, dissolve in a controlled-release, sustained-release, or other time-dependent manner. Alternatively, or additionally, the composition may comprise an aqueous solution. Such aqueous solutions or suspensions may be provided as a concentrated stock solution which is diluted prior to application, or alternatively, as a diluted solution ready-to-apply. Such compositions may be formulated in a variety of ways. They may be employed as wettable powders, granules or dusts, by mixing with various inert materials, such as inorganic minerals (silicone or silicon derivatives, phyllosilicates, carbonates, sulfates, phosphates, and the like) or botanical materials (powdered corncobs, rice hulls, walnut shells, and the like). The formulations or compositions containing genetically modified bacteria may include spreader-sticker adjuvants, stabilizing agents, other pesticidal additives, or surfactants. Liquid formulations may be employed as foams, suspensions, emulsifiable concentrates, or the like. The ingredients may include Theological agents, surfactants, emulsifiers, dispersants, or polymers.

As mentioned, the sRNA of the invention may be administered as a naked sRNA. Alternatively, the sRNA of the invention may be conjugated to a carrier known to one of skill in the art, such as a transfection agent e.g. PEI or chitosan or a protein/lipid carrier or coupled to nanoparticles. The compositions may be formulated prior to administration in an appropriate means such as lyophilized, freeze-dried, microencapsulated, desiccated, or in an aqueous carrier, medium or suitable diluent, such as saline or another buffer. Suitable agricultural carriers can be solid, semi-solid or liquid and are well known in the art. Such compositions may be considered “agriculturally-acceptable carriers”, which may cover all adjuvants, e.g., inert components, dispersants, surfactants, tackifiers, binders, etc. that are ordinarily used in pesticide formulation technology.

The invention now being generally described will be more readily understood by reference to the following examples, which are included merely for the purposes of illustration of certain embodiments of the embodiments of the present invention. The examples are not intended to limit the invention, as one of skill in the art would recognize from the above teachings and the following examples that other techniques and methods can satisfy the claims and can be employed without departing from the scope of the claimed invention. Indeed, while this invention has been particularly shown and described with references to preferred embodiments thereof, it will be understood by those skilled in the art that various changes in form and details may be made therein without departing from the scope of the invention encompassed by the appended claims

### EXAMPLES

### Example 1: Plant Endophyte Isolation and Identification

Endophytic bacterial strains were isolated from the roots of healthy hemp (Cannabis Sativa) plants. To ensure the purity of the emergent microorganisms, bacterial colonies from sterilized tissues will be passed through three rounds of single-colony isolation via streaking on lysogeny broth (LB) plates amended with filter-sterilized, antifungal agent benomyl (10 mg·L-1; Sigma-Aldrich). The purified cultures were grown together on either media by single colony spotting to identify the distinct colony types, thus, to avoid duplications. Stock cultures of pure bacteria were prepared from overnight LB broth mixed with 25% glycerol stock at 1:1 ratio and stored at −80° C.

The identity of the organisms was be established through 16S rRNA gene sequence-based homology analysis. For example, in one embodiment bacterial DNA was be isolated employing Bacterial Genomic DNA Miniprep kit in house and 16S rDNA gene amplification was be performed through PCR using universal bacterial primers [1]:

27F-AGAGTTTGATCCTGGCTCAG
and
534R-Y-GGTTACCTTGTTACGACT

The thermocycling conditions included initial one denaturation step of 94° C. for 5 min followed by 35 amplification cycles of 94° C. for 30 s, 55° C. for 40 s, 72° C. for 40 s followed by a final extension at 72° C. for 5 min. After confirming PCR amplification in a 1% agarose gel and column purification of the PCR product, the 16SrRNA gene were double end-sequenced using 27F and 534R primers. The identity of the organisms was determined by megablast analysis at the NCBI Gen Bank database and was validated through Seqmatch search at the Ribosomal Database Project. The 16s rDNA sequences analysis showed that the strains Csr-7 shared 99% homology with Pseudomonas mediterranea strain G10-2 (Accession No. MN712327.1) and Pseudomonas putida strain KDSF9 (Accession No. KF364489.1).

Phenotypic analysis was further performed on Pseudomonas sp. Csr-7 and Csr-8. Specifically, API 20 NE (V7.0, Biomerieux) analysis was performed on both Csr-7 and Csr-8 according to the company's protocol. Two technical replicates were completed for each strain. Additional oxidase tests were completed with OxiStrips (Hardy Diagnostics) according to the manufacturer's instructions. The results were as follows:

Csr-7: API generated number code: 1057555. The closest match to Csr-7 is Pseudomonas fluorescens. Csr-7 mismatches the expected results for P. fluorescens on 1 of the 21 biochemical tests (ADH; arginine dihydrolase) (95%). However, in-house sequence analysis suggests that Csr-7 is Pseudomonas mediterranea. Sequence results are typically considered more accurate methodology for species prediction. The API tests are limited by the database of tested bacterial species (P. mediterranea is not is their database). (See Table 1)

Csr-8: API generated number code: 0142457. The closest match in the API database is Pseudomonas putida. Csr-8 and P. putida match on 21 of the 21 (100%) biochemical tests. These data agree with our 16S ribosomal sequence data which also identified Csr-8 as P. putida. (See Table 1)

TABLE 1
Results from API 20NE test strips for Csr-7
and Csr-8 after 48-hour incubation period.

| | | RESULTS (48 hrs) | |
|---|---|---|---|
| TESTS | REACTION/ENZYMES | Csr-7 | Csr-8 |
| NO₃ | Reduction of nitrates to nitrites | + | − |
| | Reduction of nitrates to nitrogen | − | − |
| TRP | Indole production | − | − |
| GLU | Fermentation (glucose) | − | − |
| ADH | Arginine dihydrolase | − | + |
| URE | Urease | − | − |
| ESC | Hydrolysis (B-glucosidase) | − | − |
| GEL | Hydrolysis (protease) | + | − |
| PNPG | B-galactosidase | − | − |
| GLU | Assimilation (glucose) | + | + |
| ARA | Assimilation (arabinose) | + | − |
| MNE | Assimilation (mannose) | + | + |
| MAN | Assimilation (mannitol) | + | − |
| NAG | Assimilation (N-Acetyl- | + | − |
| | Glucosamine) | | |
| MAL | Assimilation (maltose) | − | − |
| GNT | Assimilation (potassium gluconate) | + | + |
| CAP | Assimilation (capric acid) | + | + |
| ADI | Assimilation (adipic acid) | − | − |
| MLT | Assimilation (malate) | + | + |
| CIT | Assimilation (trisodium citrate) | + | + |
| PAC | Assimilation (phenylacetic acid) | − | + |
| OX | Cytochrome oxidase | + | + |

Notably, the API 20 NE system cannot be used to identify any microorganisms not specifically in the API database (see below) or to exclude their presence. Pseudomonas species in database:
P. aeruginosa
P. fluorescens
P. luteola
P. mendocina
P. oryzihabitans
P. putida
P. stutzeri

### Example 2: Bacterial Colonization of Multiple Plant Hosts

In one embodiment, Pseudomonas sp. Csr-7 and Csr-8 were transformed with a plasmid carrying green fluorescent protein (GFP) to determine its colonization patterns in multiple plant hosts. Single colony of Csr-7 expressing GFP was introduced into a 500 mL flask containing LB with a selection antibiotic and allowed to grow at 30° C. 16-18 hours. The cultures were transferred into 175 mL conical tubes and cell pellets were collect with centrifugation at 4000 rpm for 10 minutes. The cell pellets were washed by suspending in 175 mL 1×PBS (phosphate buffered saline) and submitted to a second centrifugation at 4000 rpm for 10 minutes. The cell pellets were suspended in 200 mL 1×PBS and having an optical density (OD) absorbance 600 nm to 1.0. The bacterial suspensions were applied as a soil drench on the young seedings in the exemplary plants. Bacteria were re-isolated from surface sterilized roots, stems and leaves and plated on LB plates with a selection antibiotic with a serial dilution on a regular monthly basis. The colonies were observed on an epifluorescence microscope and submitted for 16s rDNA gene sequencing to confirm the identity of the colonies expressing GFP. The Csr-7 expressing GFP were found to colonize only in the roots on multiple exemplary plants for more than 100 days.

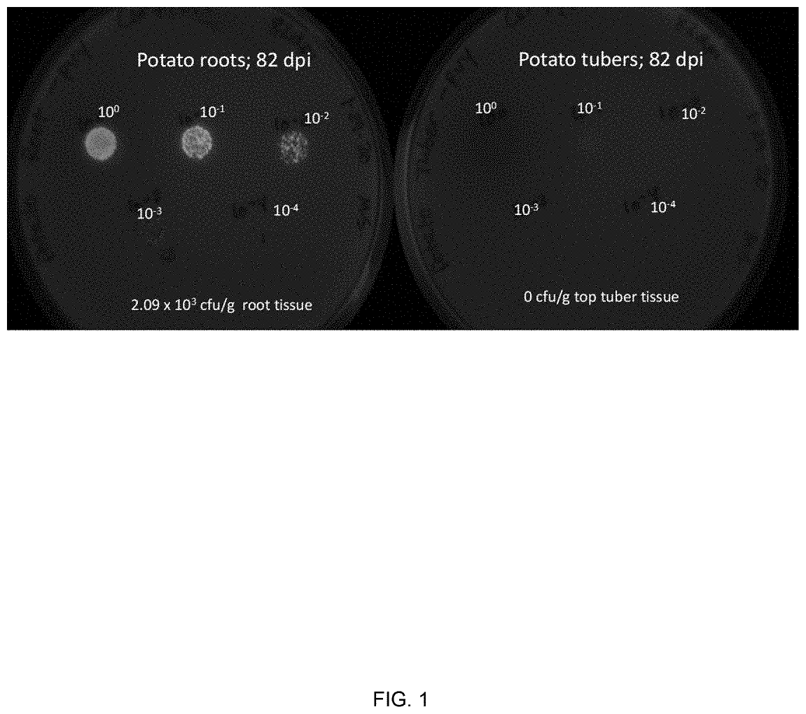

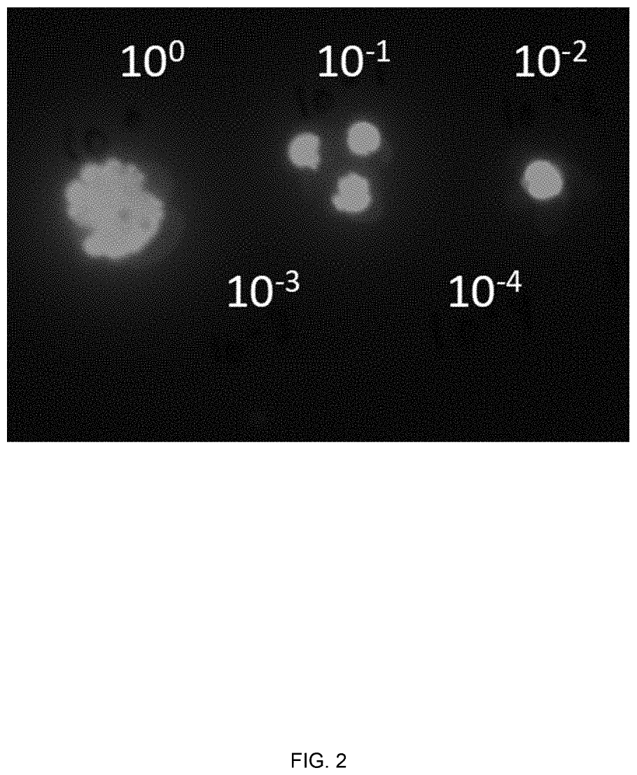

As shown in FIG. 1, Csr-7 colonies were present in potato roots, but not in the tubers at 82 days post inoculation (dpi). In potato, Csr-7 colonies were present in roots at 117 dpi with a concentration of 7.7×10² cfu/g root tissue (FIG. 2), but not stems, upper branches, flowers or fruits. Csr-7 could also colonize in the roots of hemp “Sandia Haze” at 100 dpi and in tobacco roots at least for 74 days. Csr-8 could colonize in the roots of tobacco (at least 65 dpi) and potato plants (105 dpi). Neither Csr-7 nor Csr-8 caused any adverse symptoms on the target hosts or affected the growth and development of the plants.

### Example 3: Engineered Endophyte Provides Protection Against Plant Pathogen Infection

Two hairpin RNA constructs were developed targeting 522 base pairs of Potato Virus Y (PVY) coat protein (CP) gene and they are hpPVY-CP and hpPVY-CP-RNase III E38A-R107A-R108A. These two constructs were transformed into endophyte Csr-7. RNase III E38A-R107A-R108A gene was integrated into Csr-7 bacteria genome and designated as Csr-7::RNase III E38A-R107A-R108A. Hairpin RNA construct hpPVY-CP was transformed into Csr-7:RNase III E38A-R107A-R108A. The abovementioned three strains together with wild type Csr-7 and buffer control were included in the efficacy assays using potato-PVY pathogen.

Potato (Russet Burbank) cuttings, or clones, were made from cutting from the healthy and largest apical shoots of the mother plant. Angled cuts are made (to allow as much surface area for rooting as possible) and treated with rooting hormone (bottom 2 cm) and set to root in pre-hydrated jiffy peat pellets. Once roots have developed, 10-20 days old potato cuttings were separated into treatments and inoculated with bacteria as described in Example 1. Prior to PVY infection, bacteria were confirmed colonizing in potato roots at 14-21 days after bacteria inoculation. Five grams of PVY infected tobacco leaf tissue were ground with 10 mL of inoculation buffer (1M MES pH 5.7, 0.1M Aceto, 1M CaCl₂), 1M MgSO₄) to create the inoculum and aliquoted into 200 ul to be used to inoculate each individual plant. The upper three leaves were chosen for inoculation and marked with Wite-Out® correction fluid. Three leaflets at the end of each leaf were dusted and carborundum for making abrasions on leaf surface and rubbed gently with inoculum. Carborundum was rinsed from each leaflet and plants were placed back in incubator for recovery.

Treatments of plants were fall into 6 categories: 1) control potato plants treated with 1×PBS, 2) PVY inoculation on control plants, 3) potato plants pre-inoculated with control endophytic bacterial strain Csr-7, 4) PVY inoculated plants pre-inoculated with Csr-7, 5) potato plants pre-inoculated with the endophytic bacterial strain Csr-7/hpPVY-CP, Csr-7/hpPVY-CP-RNase III E38A-R107A-R108A, and Csr-7::RNase III E38A-R107A-R108A/hpPVY-CP, respectively, and 6). PVY inoculation on plants pre-inoculated with the endophytic bacterial strains carrying each construct as described in category 5. Eight plants from each treatment were chosen for viral inoculation.

Plant leaf tissues were collected for 3 timepoints at 2-week intervals following PVY inoculation. Approximately 0.1 g of potato leaf tissue was collected into Qiagen PowerBead Tubes with 1.4 mm ceramic beads. Samples were immediately stored at −80° C. Sample tubes were placed in liquid nitrogen and then processed on the Qiagen TissueLyser II for 5 minutes at 30 Hz. Following tissue destruction, 600 μl of TRIzol Reagent (Invitrogen) was added to each tube, mixed well, and incubated for 5 minutes at room temperature. Then the Direct-zol Miniprep Plus kit (Zymo Research) was used to extract total RNA following the manufacturer's protocol. The in-column DNase I treatment was utilized, and the final RNA was resuspended in 50 μl water. To remove impurities from RNA, the OneStep PCR Inhibitor Removal kit (Zymo Research) was used following manufacturer's instructions. Concentrations were measured on the Nanodrop 2000c (Thermo Scientific), and approximately 4 μg of purified total RNA was used for cDNA synthesis. Following the company provided protocol, Takara EcoDry Premix (double primed) was utilized for cDNA synthesis. The resulting cDNA was used to quantify the relative expression of PVY against the internal potato actin gene via the comparative ΔΔCt method. qPCR reactions were setup with 10 μl iTaq Universal SYBR Green Supermix (Bio-Rad), 1 μl forward primer (10 μM), 1 μl reverse primer (10 μM), 7 μl sterile water, and 1 μl cDNA.

Primers used for potato actin [2] were:

| | |
|---|---|
| Potato_Actin97_F: | AGTATGACGAATCTGGTCCTTCTATTG |
| Potato_Actin97_R: | ACCCAACAATCAACTCTGCCCTCTC |

Primers used for PVY detection [3] were:

| | |
|---|---|
| PVY_O_F: | TGGATGGGAATGAACAAGTTGA |
| PVY_O_R: | TGCCTAAGGGTTGGTTTTGC |

Samples were processed on the Stratagene Mx3005P (Agilent Technologies) under the following conditions: 1×: 95° C. for 5 minutes; 40×: 95° C. for 10s, 60° C. for 20s, 72° C. for 20s; 1×: 95° C. for 60s, 55° C. for 30s, 95° C. for 30s.

Potato plants pre-inoculated with Csr-7/hpPVY-CP-RNase III E38A-R107A-R108A, or Csr-7: RNase III E38A-R107A-R108A/hpPVY-CP both exhibited reduced level of PVY coat protein compared with plants pre-inoculated with Csr-7/hpPVY-CP. These results indicated that modified strain with RNase III E38A-R107A-R108A either in bacteria genome or in a plasmid is more efficient than wild type strain Csr-7/hpPVY-CP at silencing the target gene of PVY.

### Example 4: Materials and Methods

Preparation and use of Electrocompetent Pseudomonas species Csr-7 and Csr-8: Pseudomonas species were streaked to isolation on LB agar. A single, well isolated colony was inoculated into LB growth media in a polypropylene cap with vent holes and a 0.22 μm hydrophobic membrane to allow for gas exchange. Cells were then grown for 16 hours at 30° C. to stationary phase. The following day 7 mL of the stationary phase culture was inoculated into a 150 mL sterile Erlenmeyer flask with 50 mL LB growth media and grown at 30° C. to optimal density at 600 nm of 0.75-0.85. Once the target optimal density is reached the cells are retained on ice and centrifugation steps be performed at 4° C. Cells are transferred to sterile 50 mL tubes in 25 mL volumes are harvested at 4000 rpm for 10 minutes. The growth media is decanted from the tube and cells are suspended gently by pipetting with ice-cold 10% sterile Glycerol solution. The cells are harvested at 4000 rpm for 10 minutes and solution decanted, followed by an additional wash with 10% Glycerol solution. After the second wash, the cells are gently resuspended in a total of 0.8 mL 10% Glycerol. The cells are aliquoted into pre-chilled sterile 1.5 mL microcentrifuge tubes. The cells may be used immediately for electroporation with plasmid or flash frozen in liquid nitrogen and immediately stored at −80° C.

Electroporation of Electrocompetent Pseudomonas species Csr-7 and Csr-8: 1 μg plasmid DNA (to not exceed a volume of more than 15 μL) is added to 50 μl of electrocompetent cells. The mixture is homogenized by gently mixing with pipette three times. The DNA cell homogenate is transferred to a pre-chilled 2 mm electroporation cuvette retained on ice. The moisture is quickly wiped from the cuvette prior to insertion into the Electroporation device. Pulse is delivered at 2,400 V, 200 Ω, 25 μF, 5 ms time constant. The time constant should be between 5.0-5.3. The cell homogenate is immediately transferred to 1 mL tryptic soy broth in a 5 mL culture tube with a two-position cap with cap loose for aerobic culturing. Incubate 2 hours at 30° C. to allow the antibiotic resistance of the plasmid to be expressed. The cell culture is plated on LB agar containing antibiotic selection using sterile glass beads at 50 and 100 μL.

### DEPOSIT INFORMATION

The bacterial strains Pseudomonas sp. Csr-7 (ATCC Patent Deposit No. PTA-126973) and Csr-8 (ATCC Patent Deposit No. PTA-126974) have been deposited in an international depository under conditions that assure that access to the culture will be available during the pendency of this patent application and any patent(s) issuing therefrom to one determined by the Commissioner of Patents and Trademarks to be entitled thereto under 37 C.F.R. 1.14 and 35 U.S.C. 122. These strains have been deposited in the American Type Culture Collection (ATCC), at 10801 University Boulevard, Manassas, Va., 20110-2209 United States of America. The date of deposit was Mar. 24, 2021.

### REFERENCES

[1] Scott, M., Rani, M., Samsatly, J., Charron, J. B. and Jabaji, S., 2018. Endophytes of industrial hemp (Cannabis sativa L.) cultivars: identification of culturable bacteria and fungi in leaves, petioles, and seeds. Canadian journal of microbiology, 64(999), pp. 1-17.
[2] Zhu, X., Richael, C.. Chamberlain, P., Busse, I S., Bussan, A. J., Jiang, J. and Bethke, P. C., 2014. Vacuolar invertase gene silencing in potato (Solanum tuberosum L.) improves processing quality by decreasing the frequency of sugar-end defects. PloS one, 9(4).
[3] Kogovšek, P., Gow, L., Pompe-Novak, M., Gruden, K., Foster, G. D., Boonham, N. and Ravnikar, M., 2008. Single-step RT real-time PCR for sensitive detection and discrimination of Potato virus Y isolates. Journal of virological methods, 149(1), pp. 1-11.

SEQUENCE LISTING

SEQ ID NO. 1
DNA
PVY coat protein (CP) sequence
Potato Virus Y
GGAAATGACACAATTGATGCAGGAGGAAGCACTAAGAAGGATGCAAAAC
AAGAGCTAGGTAGCATTCAACCAAATCTCAACAAGGAAAAGGAAAAGGA
CGTGAATGTTGGAACATCTGGAACTCATACTGTGCCACGAATTAAAGCT
ATCACGTCCAAAATGAGAATGCCCAAGAGTAAAGGTGCAACTGTACTAA
ATTTGGAACACTTACTCGAGTATGCTCCACAGCAAATTGACATCTCAAA
TACTCGAGCAACTCAATCACAGTTTGATACGTGGTATGAAGCGGTACAA
CTTGCATACGGCATAGGAGAAACTGAAATGCCAACTGTGATGAATGGGC
TTATGGTTTGGTGCATTGAAAATGGAACCTCGCCAAACATCAACGGAGT
TTGGGTTATGATGGATGGAGATGAACAAGTCGAATACCCACTGAAACCA
ATCGTTGAGAATGCAAAACCAACACTTAGGCAAATCATGGCACATTTCT
CAGATGTTGCAGAAGCGTATATAGAAATGCGCAACAAAAAGGAACCATA
TATGCCACGATATGGTTTAGTTCGTAATCTGCGCGATGGAAGTTTGGCT
CGCTATGCTTTTGACTTTTATGAGGTCACATCACGAACACCAGTGAGGG
CTAGGGAAGCGCACATTCAAATGAAGGCCGCAGCATTGAAATCAGCCCA
ACCTCGACTTTTCGGGTTGGACGGTGGCATCAGTACACAAGAGGAGAAC
ACAGAGAGGCACACCACCGAGGATGTTTCTCCAAGTATGCATACTCTAC
TTGGAGTCAAGAACATGT
SEQ ID NO. 2
DNA
RNaseIII-(rcn)-HT115-E38A-R107A-R108A
E. coli
ATGAACCCCATCGTAATTAATCGGCTTCAACGGAAGCTGGGCTACACTT
TTAATCATCAGGAACTGTTGCAGCAGGCATTAACTCATCGTAGTGCCAG
CAGTAAACACAATGCCCGCTTGGAATTTTTAGGCGACTCTATTCTGAGC
TACGTTATCGCCAATGCGCTTTATCACCGTTTCCCTCGTGTGGATGAAG
GCGATATGAGCCGGATGCGCGCCACGCTGGTCCGTGGCAATACGCTGGC
GGAACTGGCGCGCGAATTTGAGTTAGGCGAGTGCTTACGTTTAGGGCCA
GGTGAACTTAAAAGCGGTGGATTTGCCGCCGAGTCAATTCTCGCCGACA
CCGTCGAAGCATTAATTGGTGGCGTATTCCTCGACAGTGATATTCAAAC
CGTCGAGAAATTAATCCTCAACTGGTATCAAACTCGTTTGGACGAAATT
AGCCCAGGCGATAAACAAAAAGATCCGAAAACGCGCTTGCAAGAATATT
TGCAGGGTCGCCATCTGCCGCTGCCGACTTATCTGGTAGTCCAGGTACG
TGGCGAAGCGCACGATCAGGAATTTACTATCCACTGCCAGGTCAGCGGC
CTGAGTGAACCGGTGGTTGGCACAGGTTCAAGCCGTCGTAAGGCTGAGC
AGGCTGCCGCCGAACAGGCGTTGAAAAAACTGGAGCTGGAATGA
SEQ ID NO. 3
Amino Acid
RNaseIII-(rcn)-HT115-E38A-R107A-R108A-aa
E. coli
MNPIVINRLQRKLGYTFNHQELLQQALTHRSASSKHNARLEFLGDSILS
YVIANALYHRFPRVDEGDMSRMRATLVRGNTLAELAREFELGECLRLGP
GELKSGGFAAESILADTVEALIGGVFLDSDIQTVEKLILNWYQTRLDEI
SPGDKQKDPKTRLQEYLQGRHLPLPTYLVVQVRGEAHDQEFTIHCQVSG
LSEPVVGTGSSRRKAEQAAAEQALKKLELE
SEQ ID NO. 4
DNA
RNaseIII-(rcn)-HT115-Ag001-E38A-R107A-R108A
Enterobacteriaceae Ag001
ATGAATCCCATCGTAATAAATAGGCTGCAGCGTAAGCTGGGCTACACTT
TTCAACATCAGGATCTGTTGCAACAGGCATTAACCCATCGGAGTGCCAG
CAGCAAGCATAATGCCCGCTTGGAGTTTTTGGGTGACTCCATTCTCAGT
TATGTCATCGCGAATGCGCTGTATCATCGTTTTCCTCGCGTAGATGAAG
GCGACATGAGCCGCATGCGTGCGACGCTGGTGCGCGGCAATACGCTGGC
GGAAATCGCCCGCGAGTTCGAACTGGGTGAGTGTCTGCGTCTTGGGCCG
GGTGAACTGAAAAGTGGCGGTTTCGCCGCCGAGTCGATTCTTGCTGATA
CCGTGGAAGCGTTGATCGGTGGCGTCTTCCTCGACAGCGACATTCAGAA
CGTTGAGCGTTTGATTCTCTCGTGGTATCAGACCCGTCTCGACGAAATC
AGTCCAGGCGACAAGCAAAAAGATCCGAAAACGCGTCTGCAGGAGTACC
TGCAGGGTCGCCATCTGCCGCTGCCGTCGTATCTGGTGGTGCAGGTGCG
TGGTGAAGCGCACGATCAAGAATTTACCATTCACTGTCAGGTGAGTGGC
CTGCCTGAGCCTGTCGTAGGGACGGGCTCAAGCCGCCGTAAAGCGGAAC
AGGCTGCGGCTGAGCAGGCACTGAAAAAGCTGGAGCTGGAATGA
SEQ ID NO. 5
Amino Acid
RNaseIII-(rcn)-HT115-Ag001-E38A-R107A-R108A-aa
Enterobacteriaceae Ag001
MNPIVINRLQRKLGYTFQHQDLLQQALTHRSASSKHNARLEFLGDSILS
YVIANALYHRFPRVDEGDMSRMRATLVRGNTLAEIAREFELGECLRLGP
GELKSGGFAAESILADTVEALIGGVFLDSDIQNVERLILSWYQTRLDEI
SPGDKQKDPKTRLQEYLQGRHLPLPSYLVVQVRGEAHDQEFTIHCQVSG
LPEPVVGTGSSRRKAEQAAAEQALKKLELE
SEQ ID NO. 6
DNA
RNaseIII-(rcn)-HT115-Ae003-E38A-R107A-R108A
Enterobacter Ae003
ATGAACCCCATCGTAATTAATCGGCTTCAACGGAAGCTGGGCTACACTT
TTCATCATCAGGAGTTGTTGCAACAGGCATTAACCCACCGCAGTGCCAG
CAGCAAGCACAACGCCCGCCTGGAGTTTTTAGGCGACTCTATTTTAAGT
TTCGTGATTGCGAATGCGCTTTATCATCGTTTCCCGCGCGTGGATGAAG
GTGATATGAGCCGCATGCGTGCCACGCTGGTTCGGGGTAACACCCTTGC
GGAAATCGCGCGCGAATTTGAACTGGGCGAATGTCTGCGTCTTGGGCCG
GGTGAACTGAAAAGCGGCGGCTTCGCCGCCGAATCTATTCTTGCCGATA
CGGTCGAAGCATTAATTGGTGGTGTGTTCCTGGACAGCGATATCCAGAC
CGTCGAAAAGCTGATCCTGAACTGGTATCAGACCCGTCTGGACGAAATC
AGCCCGGGCGATAAACAAAAAGATCCCAAAACGCGTCTGCAGGAATATT
TGCAGGGCCGTCATCTGCCGCTGCCATCTTATCTGGTGGTGCAGGTTCG
TGGCGAAGCGCACGATCAGGAATTTACCATCCATTGCCAGGTCAGTGGC
CTGAGTGAACCGGTGGTGGGCACAGGTTCAAGCCGTCGTAAGGCTGAAC
AGGCTGCCGCCGAACAGGCGTTAAAAATGCTGGAGCTGGAATGA
SEQ ID NO. 7
Amino Acid
RNaseIII-(rcn)-HT115-Ae003-E38A-R107A-R108A-aa
Enterobacter Ae003
MNPIVINRLQRKLGYTFHHQELLQQALTHRSASSKHNARLEFLGDSILS
FVIANALYHRFPRVDEGDMSRMRATLVRGNTLAEIAREFELGECLRLGP
GELKSGGFAAESILADTVEALIGGVFLDSDIQTVEKLILNWYQTRLDEI
SPGDKQKDPKTRLQEYLQGRHLPLPSYLVVQVRGEAHDQEFTIHCQVSG
LSEPVVGTGSSRRKAEQAAAEQALKMLELE

What is claimed is:

1. A Pseudomonas bacterial strain selected from the group consisting of: Csr-7 (ATCC Patent Deposit No. PTA-126973); and Csr-8 (ATCC Patent Deposit No. PTA-126974).

2. A culture broth of the Pseudomonas bacterial strain of claim 1, a concentrate thereof, or a dry product thereof.

3. The bacterial strain of claim 1, wherein the bacterial strain is genetically engineered to express a heterologous nucleotide sequence, operably linked to a promoter, encoding a eukaryotic-like mRNA.

4. The bacterial strain of claim 1, wherein the bacterial strain is genetically engineered to express a heterologous nucleotide sequence, operably linked to a promoter, encoding an inhibitory RNA molecule that inhibits expression of at least one of the following:
an endogenous gene in a plant host;
a gene of a plant host pathogen;
a gene of a pest of a plant host; and
a gene of nematode of a plant host.

5. The bacterial strain of claim 4, wherein the heterologous nucleotide sequence encoding an inhibitory RNA molecule comprises a nucleotide sequence according to SEQ ID NO. 1.

6. The bacterial strain of claim 1, wherein the bacterial strain is genetically engineered to express a heterologous nucleotide sequence, operably linked to a promoter, encoding one or more of the following:
a heterologous double stranded RNA (dsRNA) molecule configured to inhibit expression of at least one target gene; and
a heterologous engineered RNaseIII enzyme having one or more mutations that enhance generation of small interfering RNAs (siRNAs) from catalytic cutting of the dsRNA molecule; and

7. The bacterial strain of claim 6, wherein the heterologous nucleotide sequence encoding an engineered RNaseIII enzyme comprises a polynucleotide sequence selected from the group consisting of: SEQ ID NO. 2, 4, and 6.

8. The bacterial strain of claim 6, wherein said target gene comprises at least one of the following:
an endogenous gene in a plant host;
a gene of a plant host pathogen;
a gene of a pest of a plant host; and
a gene of nematode of a plant host.

9. The bacterial strain of claim 1, wherein bacterial strain colonizes a target host plant.

10. The bacterial strain of claim 9, wherein bacterial strain colonizes the roots of the target host plant.

11. The bacterial strain of claim 1, wherein bacterial strain persists in the roots of a target host plant.

12. The bacterial strain of claim 6, wherein the dsRNA or siRNAs are produced by the bacterial strain in the roots of a target host plant and distributed through the plant's intracellular architecture initiating an RNAi response in non-root tissues of the plant.

13. The bacterial strain of claim 9, wherein said target host plant is selected from the group consisting of: a dicot plant, or a monocot plant.

14. The bacterial strain of claim 13, wherein said dicot plant, or monocot plant is selected from the group consisting of: grains, corn, wheat, rice, barley, rye, oats, sorghum, millet, sunflower, safflower, cannabis, cotton, soy, canola, alfalfa, Arabidopsis, cannabis, potato, Brassica, peanut, tobacco, tropical fruits and flowers, banana, duckweed, gladiolus, sugar cane, pineapples, dates, onions, pineapple, cashews, pistachios, flowers, ornamentals, conifers, deciduous, grapes, citrus, roses, apples, peaches, strawberries, almonds, coffee, oaks, beans, legumes, watermelon, squashes, cabbage, turnip, mustard, cacti, pecans, flax, sweet potato, soybean, coconut, avocado, maize beets, cantaloupe and vegetables.
